(19) United States
(12) Patent Application Publication
(10) Pub. No.: US 2019/0183952 A1
(43) Pub. Date: Jun. 20, 2019

(54) METHODS FOR PREPARING ACTIVE EXTRACT AND APPLICATION THEREOF

(71) Applicant: BEIJING BOXIN NATURE BIOTECH LTD., Beijing (CN)

(72) Inventors: Miao JIANG, Beijing (CN); Javier ANGULO, Madrid (ES); Shaokun TANG, Tianjin (CN); Qing JIN, Beijing (CN)

(21) Appl. No.: 16/322,160

(22) PCT Filed: Aug. 4, 2017
(86) PCT No.: PCT/CN2017/095937

§ 371 Date: Jan. 31, 2019

(30) Foreign Application Priority Data
Aug. 4, 2016 (CN) 201610634715.1

(51) Int. Cl. A61K 36/185; A61P 13/02; A61P 35/00; A61P 27/02; A61P 15/10; A61P 9/12; A61P 7/02; A61K 31/01; A61K 31/4985; A23L 33/105

(52) U.S. Cl. CPC A61K 36/185; A23V 2250/213; A61P 35/00; A61P 27/02; A61P 15/10; A61P 9/12; A61P 7/02; A61K 31/01; A61K 31/4985; A23L 33/105; A61K 2236/15; A61K 2236/333; A61K 2236/51; A61K 2236/39; A61K 2236/37; A23V 2002/00; A23V 2200/326; A23V 2200/308; A61P 13/02

(57) ABSTRACT

Disclosed is a method for preparing an active Nitraria tangutorum fruit extract comprising a solvent extraction and purificaiton process and/or a supercritical extraction process. Further disclosed are a pharmaceutical composition, a food additive and a health-care preparation each comprising the active Nitraria tangutorum fruit extract obtained by the method for preparing them, and use of the extract in the manufacture for a medicament for the treatment and/or prevention of a condition.

### TECHNICAL FIELD

The present disclosure relates to an extraction method, composition produced thereby, and a method of using the composition. In particular aspects, the present disclosure relates to a method for preparing an extract from a plant (for example, from a Nitraria fruit) and method of using the extract for a health benefit, prevention, and/or treatment of a condition, disease, or syndrome.

### BACKGROUND

Nitraria tangutorum Bobr. grows in the Qaidam Basin, which has the highest altitude among the eight largest deserts in China and is internationally known as the “purest” place. The high-altitude, intensive ultraviolet radiation, and oxygen-deficient environment all contribute to purely natural and pollution-free Nitraria plant and raw materials obtained therefrom. Nitraria fruit is a rare wild fruit. The mature Nitraria fruit is a crystal clear, pearl-like fruit. The fruit can be red or purple in color and is thus also called “red pearls in plateau.” The Nitraria fruit is rich in a variety of nutritional components and medicinally active components for a number of health benefits. The Nitraria fruit has been used by the local people for invigorating the spleen and benefiting the stomach, helping digestion, relieving uneasiness, relieving exterior syndrome, promoting lactation, and the like.

Existing methods for extracting anthocyanins and polyphenols from the Nitraria fruits and Nitraria barks involve solvent extraction, microwave- and ultrasonic-assisted extraction, resin adsorption and purification, membrane separation, and the like. In these methods, many steps are required, rendering the methods both time and labor consuming. In addition, a variety of chemical solvents are used, some of which are dangerous during the operation, toxic, and/or explosive, and some are even listed as prohibited chemicals in food due to their toxicity to humans. Furthermore, the use of some chemical solvents has adverse effect on the taste of product. Although removal of such chemical solvents can be performed subsequently, residue chemicals are hard to remove and it is difficult to ensure the product is safe for human use. The solvent extraction method is non-green, and the subsequent removal of solvents requires high temperature which will destroy the functional and nutritional components. Furthermore, the content and yield of the target ingredients have not been mentioned in most art methods.

In addition, studies show that, for the functional components of natural plants, after many times of purifications and extractions, the functional activity will decrease although the concentration of a single component may be increased. It is indicated that the nutritional, health-care and medicinal functions of natural plants are often more related to the plurality of components in the plants instead of a single component of the plants.

Therefore, there is a need for an improved method of extracting plant ingredients, for example, for use in improving human health and preventing and/or treating a disease or condition.

### SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

There is great value for developing Nitraria fruit products due to its green property and medicinal function. Studies show that the functional and nutritional components of the Nitraria fruit have the following advantages: they help resist oxidation, remove free radicals, resist damages caused by UV, remove metal residues in the body, regulate functions of the immune system, resist mutation, prevent the occurrence of diseases such as cancers, regulate the blood lipid, dilate the blood vessels, regulate the male and female hormones, regulate the blood glucose, and relieve lower urinary tract symptoms due to benign prostatic hypertrophy, and the like.

In one aspect, disclosed herein is a method for obtaining an extract from Nitraria tangutorum Bobr. In one aspect, the method comprises mixing a sample of Nitraria tangutorum Bobr. fruit with an alcohol (e.g., ethanol) solution, e.g., from about 30% (v/v) to about 95% (v/v), such as about 65% (v/v). In another aspect, the method comprises incubating the mixture under a temperature between about 10° C. and about 60° C., such as between about room temperature (e.g., about 18° C.) and about 55° C. In yet another aspect, the method comprises obtaining a liquid phase sample from the mixture. In one aspect, the method comprises filtering the liquid phase sample, removing the alcohol from the liquid phase sample, and/or concentrating the liquid phase sample. In any of the preceding embodiments, the remaining sample can comprise a solid, semi-solid, and/or liquid matter. In one other aspect, the method comprises allowing evaporation of the alcohol and/or water from the liquid phase sample. In any of the preceding embodiments, the resulting sample after evaporation can comprise an extract comprising one or more ingredients from Nitraria tangutorum Bobr.

In one aspect, the method comprises: (1) mixing a sample of Nitraria tangutorum Bobr. fruit with an alcohol (e.g., ethanol) solution, e.g., from about 30% (v/v) to about 95% (v/v), such as about 65% (v/v); (2) incubating the mixture under a temperature between about 10° C. and about 60° C., such as between about room temperature (e.g., about 18° C.) and about 55° C.; (3) obtaining a liquid phase sample from the mixture, and optionally filtering the liquid phase sample, removing the alcohol from the liquid phase sample, and/or concentrating the liquid phase sample, optionally the remaining sample comprising solid, semi-solid, and/or liquid matter; and (4) allowing evaporation of the alcohol and/or water from the liquid phase sample, and the resulting sample after evaporation comprises an extract comprising one or more ingredients from Nitraria tangutorum Bobr.

In any of the preceding embodiments, in the mixing step, the ratio between the sample weight and the alcohol solution volume can be between about (1 g):(3 mL) and about (1 g):(10 mL), optionally between about (1 g):(3 mL) and about (1 g):(5 mL).

In any of the preceding embodiments, the incubating step can be carried out for between about 1 hour and about 2 hours. In any of the preceding embodiments, the incubating step can be carried out for more than about 2 hours.

In any of the preceding embodiments, the incubating step can be carried out while stirring the mixture, e.g., for extraction of the ingredient into the liquid phase sample.

In any of the preceding embodiments, in the obtaining step, the liquid phase sample can be extracted from the mixture. In any of the preceding embodiments, the remaining sample can comprise mostly solid matter. For example, at least about 50%, at least about 55%, at least about 60%, at least about 65%, at least about 70%, at least about 75%, at least about 80%, at least about 85%, at least about 90%, at least about 95%, or at least about 99% of the mass of the remaining sample is solid matter.

In any of the preceding embodiments, the sample of Nitraria tangutorum Bobr. fruit can comprise an intact fruit, flesh of the fruit, fruit pulp, a seed, a fresh fruit, a dried fruit, a chilled fruit, a frozen fruit, a preserved fruit, a milled fruit, a minced fruit, a crushed fruit, a granulated fruit, a powered fruit, or any combination thereof.

In any of the preceding embodiments, the sample of Nitraria tangutorum Bobr. fruit can be a dried sample, a semi-wet sample, or a wet sample. In any of the preceding embodiments, the sample of Nitraria tangutorum Bobr. fruit can comprise about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 99.5%, or about 99.9% water by weight.

In any of the preceding embodiments, the method can further comprise obtaining the sample of Nitraria tangutorum Bobr. fruit before the mixing step.

In any of the preceding embodiments, the method can further comprise cutting, shredding, mincing, or milling the sample of Nitraria tangutorum Bobr. fruit before the mixing step.

In any of the preceding embodiments, the method can further comprise purifying or isolating the extract and/or the ingredient from the resulting sample after evaporation.

In any of the preceding embodiments, the method can further comprise drying the resulting sample after evaporation. In any of the preceding embodiments, a powder extract comprising the ingredient can be obtained.

In any of the preceding embodiments, steps (1)-(3) can be repeated one, two, or more times before step (4).

In any of the preceding embodiments, after obtaining the liquid phase sample, the method can further comprise obtaining from the mixture a remaining sample which comprises mostly solid matter, and the mixture is a first mixture. In any of the preceding embodiments, the method can further comprise mixing the remaining sample with an alcohol (e.g., ethanol) solution, e.g., from about 30% (v/v) to about 95% (v/v), such as about 65% (v/v), to obtain a second mixture. In any of the preceding embodiments, the ratio between the weight of the remaining sample and the alcohol solution volume can be between about (1 g):(3 mL) and about (1 g):(10 mL), for example, between about (1 g):(3 mL) and about (1 g):(5 mL). In any of the preceding embodiments, the method can further comprise incubating the second mixture under a temperature between about room temperature (e.g., about 18° C.) and about 55° C. In any of the preceding embodiments, the method can further comprise obtaining a second liquid phase sample from the second mixture, and the liquid phase sample from the first mixture is a first liquid phase sample. In any of the preceding embodiments, the method can further comprise combining the first and second liquid phase samples. In any of the preceding embodiments, the method can further comprise filtering the combined liquid phase sample. In any of the preceding embodiments, the method can further comprise allowing evaporation of water and/or alcohol from the combined liquid phase sample, and the resulting sample after evaporation comprises the extract comprising one or more ingredients from Nitraria tangutorum Bobr.

In any of the preceding embodiments, after obtaining the liquid phase sample, the method can further comprise: (a) obtaining from the mixture a remaining sample which comprises mostly solid matter, and the mixture is a first mixture; (b) mixing the remaining sample with an alcohol (e.g., ethanol) solution, e.g., from about 30% (v/v) to about 95% (v/v), such as about 65% (v/v), and optionally the ratio between the weight of the remaining sample and the alcohol solution volume is between about (1 g):(3 mL) and about (1 g):(10 mL) and optionally between about (1 g):(3 mL) and about (1 g):(5 mL), to obtain a second mixture; (c) incubating the second mixture under a temperature between about room temperature (e.g., about 18° C.) and about 55° C.; (d) obtaining a second liquid phase sample from the second mixture, and the liquid phase sample from the first mixture is a first liquid phase sample; (e) combining the first and second liquid phase samples, and optionally filtering the combined liquid phase sample; and (f) allowing evaporation from the combined liquid phase sample, and the resulting sample after evaporation comprises the extract comprising one or more ingredients from Nitraria tangutorum Bobr.

In one embodiment, steps (a)-(d) are repeated one, two, or more times before step (e). In another embodiment, steps (a)-(e) are repeated one, two, or more times before step (f).

In any of the preceding embodiments, the method can further comprise a pressure treatment and/or ultra-sonication during the mixing, incubating, and/or obtaining step. In one aspect, the pressure is between about 5 MPa and about 150 MPa, e.g., 30 MPa or 50 MPa. In any of the preceding embodiments, the ultra-sonication can have a power between about 100 W and about 10,000 W, e.g., about 300 W, 400 W, 500 W, 600 W, 700 W, 800 W, or 1000 W.

In any of the preceding embodiments, the method can further comprise rotatable evaporating the liquid phase sample at a temperature of between about 20° C. and about 70° C. (such as about 55° C.) and/or a reduced pressure (such as between about −5 MPa and about 5 MPa), e.g., in order to remove a solvent in the liquid phase sample.

In any of the preceding embodiments, the method can further comprise passing the liquid phase sample through a first chromatography column. In any of the preceding embodiments, the first chromatography column can comprise a macroporous resin adsorption column. In any of the preceding embodiments, passing the liquid phase sample through the first chromatography column can remove one or more sugars or polysaccharides from the liquid phase sample.

In any of the preceding embodiments, the method can further comprise passing the liquid phase sample through a second chromatography column. In any of the preceding embodiments, the second chromatography column can comprise a macroporous resin adsorption column. In any of the preceding embodiments, passing the liquid phase sample through the second chromatography column can increase the anthocyanin and/or polyphenol concentration in the liquid phase sample.

In any of the preceding embodiments, the first and second chromatography columns can be the same or different.

In any of the preceding embodiments, before the chromatography, a part or substantially all of the alcohol can be removed (e.g., by drying or rotatable evaporating the alcohol) from the liquid phase sample, which is then re-dissolved in water. As used herein, substantially all of the alcohol can include about 60%, about 65%, about 70%, about 75%, about 80%, about 85%, about 90%, about 95%, about 99%, or about 99.9% of the alcohol.

In any of the preceding embodiments, the chromatography can comprise eluting the column, e.g., with an alcohol solution of about 50% (v/v) to about 100% (v/v), such as about 95% (v/v).

In any of the preceding embodiments, the allowing step can comprise freeze-drying (lyophilization) and/or spray-drying the resulting sample to obtain the extract.

In another aspect, disclosed herein is a method for obtaining an extract from Nitraria tangutorum Bobr., comprising: (1) extracting a sample of Nitraria tangutorum Bobr. fruit in a first supercritical fluid extraction device, to obtain an oil-like extract and a remaining sample; (2) mixing the remaining sample with an entrainer and extracting the mixture in a second supercritical fluid extraction device, to obtain a liquid phase sample; and (3) allowing evaporation from the liquid phase sample, and the resulting sample after evaporation comprises an extract comprising one or more ingredients from Nitraria tangutorum Bobr.

In some embodiments, the method comprises extracting a sample of Nitraria tangutorum Bobr. fruit in a first supercritical fluid extraction device, and an oil-like extract and a remaining sample are obtained as a result of this step. In any of the preceding embodiments, the method can further comprise mixing the remaining sample with an entrainer (also known as co-solvent) and extracting the mixture in a second supercritical fluid extraction device, and a liquid phase sample is obtained as a result of this step.

In any of the preceding embodiments, the first supercritical extraction can be carried out under a pressure between about 15 MPa and about 55 MPa, for example, about 20 MPa and about 35 MPa.

In any of the preceding embodiments, the first supercritical extraction can be carried out under a temperature between about 35° C. and about 55° C.

In any of the preceding embodiments, the first supercritical extraction can be carried out under a supercritical fluid flow rate between 0.5 L/min and about 3 L/min (such as 2 L/min), for example, between about 1 L/min and about 3 L/min.

In any of the preceding embodiments, the first supercritical extraction can be carried out for between about 1 hour and about 3 hours, for example, between about 2 hours and about 3 hours.

In any of the preceding embodiments, the second supercritical extraction can be carried out under a pressure between about 20 MPa and about 35 MPa.

In any of the preceding embodiments, the second supercritical extraction can be carried out under a temperature between about 35° C. and about 55° C.

In any of the preceding embodiments, the second supercritical extraction can be carried out under a supercritical fluid flow rate of about 1 L/min.

In any of the preceding embodiments, the second supercritical extraction can be carried out under an entrainer flow rate between about 0.2 mL/min and about 1.0 mL/min.

In any of the preceding embodiments, the second supercritical extraction can be carried out for between about 1 hour and about 3 hours.

In any of the preceding embodiments, the remaining sample in the mixing step can be a defatted remaining sample.

In any of the preceding embodiments, between about 10 g and about 500 kg (such as about 10 g, 200 kg, or 500 kg) of the sample of Nitraria tangutorum Bobr. fruit can be used for each extraction.

In any of the preceding embodiments, the supercritical fluid can comprise CO₂. In any of the preceding embodiments, the first and second supercritical fluid extraction devices can be the same or different. In any of the preceding embodiments, the entrainer can comprise an alcohol (e.g., ethanol) and/or water.

In any of the preceding embodiments, the entrainer can comprise between about 35% (v/v) and about 95% (v/v) of an alcohol such as ethanol.

In any of the preceding embodiments, the ratio between the volume of the entrainer and the weight of the remaining sample in the mixing step can be about (1 mL):(1 g), or less than about (1 mL):(1 g), such as less than about (0.1 mL):(1 g), between about (0.1 mL):(1 g) and about (0.5 mL):(1 g), or between about (0.5 mL):(1 g) and about (1 mL):(1 g).

In any of the preceding embodiments, the remaining sample can be partially or fully infiltrated with the entrainer in the mixing step. In particular embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the entrainer has infiltrated the remaining sample. In particular embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the volume or mass of the remaining sample is infiltrated with the entrainer.

In any of the preceding embodiments, in the mixing step, the remaining sample, after being infiltrated with the entrainer and prior to the second supercritical fluid extraction, can be statically soaked in the supercritical fluid (e.g., CO₂), e.g., for about 30 minutes.

In any of the preceding embodiments, between about 5 g and about 250 kg (such as about 5 g, 100 kg, or 250 kg) of the remaining sample in the mixing step can be used for the second supercritical fluid extraction.

In any of the preceding embodiments, the sample of Nitraria tangutorum Bobr. fruit can comprise an intact fruit, flesh of the fruit, fruit pulp, a seed, a fresh fruit, a dried fruit, a chilled fruit, a frozen fruit, a preserved fruit, a milled fruit, a minced fruit, a crushed fruit, a granulated fruit, a powered fruit, or any combination thereof. In any of the preceding embodiments, the sample of Nitraria tangutorum Bobr. fruit can be a dried sample, a semi-wet sample, or a wet sample. In any of the preceding embodiments, the sample of Nitraria tangutorum Bobr. fruit can comprise about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 99.5%, or about 99.9% water by weight.

In any of the preceding embodiments, the method can further comprise obtaining the sample of Nitraria tangutorum Bobr. fruit before the extracting step.

In any of the preceding embodiments, the method can further comprise cutting, shredding, mincing, or milling the sample of Nitraria tangutorum Bobr. fruit before the extracting step.

In any of the preceding embodiments, the method can further comprise purifying or isolating the extract and/or the ingredient from the resulting sample after evaporation.

In any of the preceding embodiments, the method can further comprise drying the resulting sample after evaporation.

In any of the preceding embodiments, the method can further comprise obtaining a powder extract or a semi-solid extract comprising the ingredient.

In any of the preceding embodiments, the drying step can comprise freeze-drying (lyophilization) and/or spray-drying.

In any of the preceding embodiments, the method can further comprise rotatably evaporating the liquid phase sample before drying, at a temperature between about 35° C. and about 55° C. (e.g., at about 50° C.), e.g., in order to remove a solvent in the liquid phase sample. In any of the preceding embodiments, the method can further comprise rotatable evaporating the liquid phase sample before drying, under a pressure of about −0.09 MPa, e.g., in order to remove a solvent in the liquid phase sample. In any of the preceding embodiments, the drying can be carried out at a temperature of about −50° C. and/or under a pressure of about 5 Pa.

In any of the preceding embodiments, the one or more ingredients can comprise one or more anthocyanins and/or one or more polyphenols.

In yet another aspect, provided herein is a liquid phase sample, a remaining sample, a resulting sample, an extract, and/or one or more ingredients from Nitraria tangutorum Bobr. produced by the method according to any one of the preceding embodiments.

In still another aspect, provided herein is a liquid phase sample, a remaining sample, a resulting sample, an extract, and/or one or more ingredients from Nitraria tangutorum Bobr. produced by the method according to any one of the preceding embodiments, for use in the treatment and/or prevention of a condition or disease in a subject in need thereof, optionally without adversely affecting an arterial pressure such as the mean arterial pressure and/or heart rate of the subject.

In one aspect, provided herein is use of the liquid phase sample, the remaining sample, the resulting sample, the extract, and/or the one or more ingredients from Nitraria tangutorum Bobr. of any one of the preceding embodiments, in the manufacture of a medicament for treating and/or preventing a condition or disease in a subject in need thereof.

In another aspect, provided herein is an oil-like extract, a remaining sample, a liquid phase sample, a resulting sample, an extract, and/or one or more ingredients from Nitraria tangutorum Bobr. produced by the method according to any one of the preceding embodiments. Also provided herein is the oil-like extract, the remaining sample, the liquid phase sample, the resulting sample, the extract, and/or the one or more ingredients from Nitraria tangutorum Bobr. for use in the treatment and/or prevention of a condition or disease in a subject in need thereof.

In one aspect, provided herein is use of the oil-like extract, the remaining sample, the liquid phase sample, the resulting sample, the extract, and/or the one or more ingredients from Nitraria tangutorum Bobr. of any one of the preceding embodiments, in the manufacture of a medicament for treating and/or preventing a condition or disease in a subject in need thereof.

In yet another aspect, the present disclosure relates to a pharmaceutical composition comprising the liquid phase sample, the remaining sample, the resulting sample, the extract, and/or the one or more ingredients from Nitraria tangutorum Bobr. of any one of the preceding embodiments, and optionally a pharmaceutically acceptable excipient and/or diluent.

In yet another aspect, the present disclosure relates to a pharmaceutical composition comprising the oil-like extract, the remaining sample, the liquid phase sample, the resulting sample, the extract, and/or the one or more ingredients from Nitraria tangutorum Bobr. of any one of the preceding embodiments, and optionally a pharmaceutically acceptable excipient and/or diluent.

In any of the preceding embodiments, the pharmaceutical composition can further comprise lycopene or a composition comprising lycopene, saw palmetto or an extract thereof, pumpkin seed or an extract thereof (such as a protein extract), Selenium (Se), Lyceum ruthenicum or an extract thereof, black tomato or an extract thereof, lentinan, Pleurotus ostreatus polysaccharide, agaric polysaccharide,Flammulina velutipes polysaccharide, spirulina or an extract thereof, lutein, zeaxanthin, and/or astaxanthin.

In any of the preceding embodiments, the pharmaceutical composition can be in a human dosage form.

In any of the preceding embodiments, the pharmaceutical composition can be for use in the treatment and/or prevention of a condition and/or disease in a subject in need thereof, optionally without adversely affecting an arterial pressure such as the mean arterial pressure and/or heart rate of the subject. In any of the preceding embodiments, the pharmaceutical composition can be for use in the treatment and/or prevention of a fundus lesion and/or a stomach illness. In one aspect, the condition and/or disease comprises a lower urinary tract symptom due to benign prostatic hypertrophy, macular degeneration, and a cancer. In one embodiment, the macular degeneration is associated with radiation (e.g., from a radioactive material or from an ultraviolet light). In another embodiment, the macular degeneration is age-related.

In one embodiment, the cancer is prostate cancer. In any of the preceding embodiments, the cancer can be sensitive to an androgen, such as testosterone or dihydrotestosterone. In any of the preceding embodiments, the cancer can be insensitive to an androgen, such as testosterone or dihydrotestosterone.

In any of the preceding embodiments, the liquid phase sample, the remaining sample, the resulting sample, the extract, and/or the one or more ingredients from Nitraria tangutorum Bobr. in the pharmaceutical composition can exert an anti-cancer effect, such as an anti-proliferative effect, optionally without adversely affecting an arterial pressure such as the mean arterial pressure and/or heart rate of the subject. In one embodiment, the anti-proliferative effect is independent of androgen signaling.

In some embodiments, the liquid phase sample, the remaining sample, the resulting sample, the extract, and/or the one or more ingredients from Nitraria tangutorum Bobr. in the pharmaceutical composition do not interfere with androgen-stimulated prostate specific antigen (PSA) production in the cancer.

In any of the preceding embodiments, the liquid phase sample, the remaining sample, the resulting sample, the extract, and/or the one or more ingredients from Nitraria tangutorum Bobr. in the pharmaceutical composition can relax a tissue or an organ. In some embodiments, the organ is a prostate or bladder and the tissue is a human prostate tissue or a human bladder neck.

In any of the preceding embodiments, the tissue or organ can be from a subject suffering from a lower urinary tract symptom due to benign prostatic hypertrophy, or a subject suspected of suffering from the lower urinary tract symptom due to benign prostatic hypertrophy, wherein the benign prostatic hypertrophy is optionally induced by an androgen.

In any of the preceding embodiments, the tissue or organ can be relaxed due to stimulation of NO and/or cGMP (nitric oxide and/or cyclic GMP) production.

In any of the preceding embodiments, the method can further comprise a PDE5 inhibitor such as tadalafil.

In any of the preceding embodiments, the lycopene or a composition or extract comprising lycopene can have an anti-cancer effect, such as an anti-proliferative effect. In one embodiment, the lycopene or composition or extract comprising lycopene inhibits or reduces PSA production in a cancer such as prostate cancer. In one embodiment, the anti-proliferative effect is dependent on androgen signaling, and lycopene inhibits androgen-induced proliferation of an androgen-sensitive cancer cell but not proliferation of an androgen-insensitive cancer cell.

In any of the preceding embodiments, the lycopene or a composition or extract comprising lycopene can enhance an anti-cancer effect of the liquid phase sample, the remaining sample, the resulting sample, the extract, and/or the one or more ingredients from Nitraria tangutorum Bobr. In one embodiment, the anti-cancer effect is an anti-proliferative effect on an androgen-sensitive cancer cell, such as a human prostate cancer cell.

In any of the preceding embodiments, the lycopene or a composition or extract comprising lycopene can relax a tissue or an organ. In one embodiment, the lycopene or a composition or extract comprising lycopene does not interfere with relaxation of the organ or tissue caused by the liquid phase sample, the remaining sample, the resulting sample, the extract, and/or the one or more ingredients from Nitraria tangutorum Bobr. in the pharmaceutical composition.

In any of the preceding embodiments, the pharmaceutical composition can be for improving a vascular function, blood circulation, a cerebral function, and/or an immune function, and/or for preventing and/or alleviating a symptom and/or consequence of erectile dysfunction, hypertension, arteriosclerosis, thrombosis, fatigue, cerebral apoplexy, and/or stroke. In any of the preceding embodiments, the pharmaceutical composition can be for anti-thrombosis.

In one embodiment, the pharmaceutical composition stimulates NO and/or cGMP production and optionally comprises a PDE5 inhibitor such as tadalafil.

In any of the preceding embodiments, the pharmaceutical composition can relax a tissue or an organ. In one embodiment, the organ is a penis and the tissue is a smooth muscle, such as a corpus cavernosum.

In any of the preceding embodiments, the pharmaceutical composition can be for alleviating a side effect of a therapy, such as treatment with an anticancer agent.

In any of the preceding embodiments, the pharmaceutical composition can be prepared in a form selected from the group consisting of a liquid, a powder a tablet, a granule, a pill, a capsule (e.g., a hard capsule or a soft capsule), an oral cream, a paste, a decoction, a syrup, a wine, a distillate, and any combination thereof.

In any of the preceding embodiments, the pharmaceutical composition can be in a dosage form for oral, gastrointestinal, topical, mucosal, intravenous, intradermal, subcutaneous, or intramuscular administration.

In another aspect, disclosed herein is a method of treating and/or preventing a condition and/or disease in a subject in need thereof, comprising administering to the subject a pharmaceutically effective dose of: (i) the liquid phase sample, the remaining sample, the resulting sample, the extract, and/or the one or more ingredients from Nitraria tangutorum Bobr. of any of the preceding embodiments; (ii) the oil-like extract, the remaining sample, the liquid phase sample, the resulting sample, the extract, and/or the one or more ingredients from Nitraria tangutorum Bobr. of any of the preceding embodiments; and/or (iii) the pharmaceutical composition according to any of the preceding embodiments.

In any of the preceding embodiments, the method can be used in combination with another therapy or regimen for treating and/or preventing the condition and/or disease.

In any of the preceding embodiments, the method can be used before, during, and/or after the other therapy or regimen, or in an alternating fashion with the other therapy or regimen.

In any of the preceding embodiments, the method can further comprise administering to the subject a pharmaceutically effective dose of lycopene or a composition or extract comprising lycopene.

In any of the preceding embodiments, the method can further comprise administering to the subject a pharmaceutically effective dose of a PDE5 inhibitor such as tadalafil.

In any of the preceding embodiments of the method, the condition and/or disease can be selected from the group consisting of a lower urinary tract symptom due to benign prostatic hypertrophy (e.g., BPH/LUTS), macular degeneration, a cancer such as prostate cancer (including androgen-sensitive or androgen-insensitive prostate cancer) or bladder cancer, erectile dysfunction, hypertension, arteriosclerosis, thrombosis, fatigue, cerebral apoplexy, and stroke, or any combination thereof.

In any of the proceeding embodiments of the method, the administration can be done without adversely affecting_an arterial pressure such as the mean arterial pressure and/or heart rate of the subject.

In another aspect, the present disclosure relates to a food additive comprising: (i) the liquid phase sample, the remaining sample, the resulting sample, the extract, and/or the one or more ingredients from Nitraria tangutorum Bobr. of any of the preceding embodiments; (ii) the oil-like extract, the remaining sample, the liquid phase sample, the resulting sample, the extract, and/or the one or more ingredients from Nitraria tangutorum Bobr. of any of the preceding embodiments; and/or (iii) the pharmaceutical composition according to any of the preceding embodiments.

In another aspect, the present disclosure relates to a health supplement comprising: (i) the liquid phase sample, the remaining sample, the resulting sample, the extract, and/or the one or more ingredients from Nitraria tangutorum Bobr. of any of the preceding embodiments; (ii) the oil-like extract, the remaining sample, the liquid phase sample, the resulting sample, the extract, and/or the one or more ingredients from Nitraria tangutorum Bobr. of any of the preceding embodiments; and/or (iii) the pharmaceutical composition according to any of the preceding embodiments.

In any of the preceding embodiments, the food additive and/or health supplement can be in a form selected from the group consisting of a liquid, a powder a tablet, a granule, a pill, a capsule (e.g., a hard capsule or a soft capsule), an oral cream, a paste, a decoction, a syrup, a wine, a distillate, and any combination thereof.

### BRIEF DESCRIPTION OF THE DRAWINGS

In order to more clearly illustrate the embodiments of the present disclosure, the drawings are briefly described below. It is apparent that the drawings in the following description are embodiments of the present disclosure, and other drawings may be obtained based on the present disclosure by a person with ordinary skill in the technical field without creative effort.

FIG. 1 is a chart showing the influence of the CO₂ flow rates on the yield during the supercritical extraction for extracting from the Nitraria fruit, according to one aspect of the present disclosure.

FIG. 2 shows an oil-like extract and a defatted composition obtained by the supercritical extraction from the Nitraria fruit, according to one aspect of the present disclosure.

FIG. 3 shows extracted solutions obtained from supercritical extraction of the defatted composition, in the method for preparing an active Nitraria fruit extract, according to one aspect of the present disclosure.

FIG. 4 shows a powder extract obtained by drying the extracted solution obtained from FIG. 3, according to one aspect of the present disclosure.

FIG. 5 shows a flowchart for method for preparing an active Nitraria fruit extract using solvent extraction, according to one aspect of the present disclosure.

FIG. 6 shows a flowchart for method for preparing an active Nitraria fruit extract using supercritical extraction or solvent extraction, according to one aspect of the present disclosure. The steps for the supercritical extraction and the resultant products, as well as the steps for the solvent extraction and the resultant products, are shown in the chart.

FIG. 7 shows the effects of N. tangutorum Bobr extract (NtB) on proliferation of androgen-sensitive human prostate cancer cell line, LNCaP. Data are expressed as mean±SEM of the percentage of absorbance at 490 nm (XTT assay) obtained in control conditions. * p<0.05, ** p<0.01, *** p<0.001 vs. vehicle; †† p<0.01, ††† p<0.001 vs. dihydrotestosterone (DHT) or testosterone (T) by one-factor ANOVA followed by Student-Newmann-Keuls test.

FIG. 8 shows the effects of N. tangutorum Bobr extract (NtB) on proliferation of androgen-independent human prostate cancer cell line, PC-3. Data are expressed as mean±SEM of the percentage of absorbance at 490 nm (XTT assay) obtained in control conditions. * p<0.05, ** p<0.01, *** p<0.001 vs. vehicle; † p<0.05, †† p<0.01, ††\ p<0.001 vs. DHT or T by one-factor ANOVA followed by Student-Newmann-Keuls test.

FIG. 9 shows the effects of N. tangutorum Bobr extract (NtB) on androgen-stimulated prostatic specific antigen (PSA) production by human prostate cancer cell line, LNCaP. Data are expressed as mean±SEM of the concentration of PSA (ng/ml) normalized by the cell content determined by crystal violet staining. * p<0.05, ** p<0.01, *** p<0.001 vs. only vehicle-treated cells by one-factor ANOVA followed by Student-Newmann-Keuls test.

FIG. 10 shows relaxation induced by N. tangutorum Bobr extract (NtB) on norepinephrine (NE)-contracted human prostate and bladder neck strips from patients with BPH/LUTS. Data are expressed as mean±SEM of the reversion of NE-induced contraction. * p<0.05, ** p<0.01, *** p<0.001 vs. vehicle by Student t-test.

FIG. 11 shows cyclic GMP (cGMP) accumulation induced by exposure to N. tangutorum Bobr extract (NtB; 30 mg/ml) in human prostate and bladder neck from patients with BPH/LUTS. Data are expressed as mean±SEM of cGMP content (pmol) normalized by mg of protein of the tissue. * p<0.05 vs. vehicle by Student t-test.

FIG. 12 shows effects of N. tangutorum Bobr extract (NtB; 10 mg/ml) on tadalafil induced relaxations in human prostate and bladder neck from patients with BPH/LUTS. Data are expressed as mean±SEM of the reversion of NE-induced contraction. *** p<0.001 vs. vehicle by two-factors ANOVA.

FIG. 13 shows tracings of intravesical pressure (IVP) recordings during cystometries (20 min NaCl 0.9% infusion, horizontal black lines) performed in rats with testosterone-induced BPH before (left tracings) and after intraduodenal (i.d.) administration of NtB (100 mg/kg). Bladder activity is reduced after NtB administration.

FIGS. 14A-14G show effects of intraduodenal administration of N. tangutorum Bobr extract (NtB; 100 mg/kg) on urodynamic parameters in rats with testosterone-induced BPH. Data were collected from seven different rats and are expressed as mean±SEM. * p<0.05 vs. control (before NtB administration) by paired Student t-test. Graphs show the effects of NtB on number of micturitions in 20 min infusion (FIG. 14A), micturition volume (FIG. 14B), time of infusion for producing first micturition (FIG. 14C), residual volume contained in the bladder (FIG. 14D); intravesical pressure increase (ΔIVP) during micturition (FIG. 14E), threshold of IVP for developing micturition reflex (FIG. 14F), and total bladder activity during infusion (area under the curve of the IVP) (FIG. 14G).

FIGS. 15A-15F show effects of two-weeks daily oral administration of N. tangutorum Bobr extract (NtB; 30 mg/kg) on urodynamic parameters and prostate hypertrophy in rats with testosterone-induced BPH. Data were collected from seven rats treated with vehicle (tap water) and five rats treated with NtB extract and are expressed as mean±SEM. * p<0.05 vs. vehicle-treated rats by unpaired Student t-test. Graphs show the effects of chronic NtB on number of micturitions in 20 min infusion (FIG. 15A), micturition volume (FIG. 15B), time of infusion for producing first micturition (FIG. 15C), residual volume contained in the bladder (FIG. 15D); intravesical pressure increase (ΔIVP) during micturition (FIG. 15E), threshold of IVP for developing micturition reflex (FIG. 15F), total bladder activity during infusion (area under the curve of the IVP) (FIG. 15G).and prostate weight/body weight ratio (FIG. 15H)

FIGS. 16A-16F show effects of lycopene (Lyc) on proliferation of androgen-independent human prostate cancer cell line, PC-3 (A, C, E) and androgen-sensitive human prostate cancer cell line LNCaP (B, D, F) in unstimulated conditions (A, B) or after stimulation with testosterone (T, 40 nM) (C, D) or dihydrotestosterone (DHT, 40 nM) (E, F). Data are expressed as mean±SEM of the percentage of absorbance at 490 nm (XTT assay) obtained in control conditions. Numbers of different cultures are in parenthesis. * p<0.05, ** p<0.01 vs. vehicle; † p<0.05, †† p<0.01 vs. DHT or T by one-factor ANOVA followed by Student-Newmann-Keuls test.

FIGS. 17A-17D show influence of lycopene (LYC; 0.1 and 0.25 mg/ml) on anti-proliferative effects of NtB (1, 3 and 10 mg/ml) in androgen-independent human prostate cancer cell line, PC-3 (A, C) and androgen-sensitive human prostate cancer cell line LNCaP (B, D) after stimulation with testosterone (T, 40 nM) (A, B) or dihydrotestosterone (DHT, 40 nM) (C, D). Data are expressed as mean±SEM of the percentage of absorbance at 490 nm (XTT assay) obtained in cells treated only with T or DHT. n indicates number of different cultures. * p<0.05, ** p<0.01, *** p<0.001 vs. T or DHT; † p<0.05, †† p<0.01 vs. NtB alone by one-factor ANOVA followed by Student-Newmann-Keuls test.

FIGS. 18A-16B show effects of lycopene on androgen-stimulated PSA production in human prostate cancer cells stimulated with testosterone (T) (FIG. 18A) or dihydrotestosterone (DHT) (FIG. 18B). Data are expressed as mean±SEM of ng/ml of PSA normalized by number of cells in each well determined by absorbance at 590 nm after crystal violet staining. Number of determinations are in parenthesis. * p<0.05 vs. control (without T or DHT). by one factor ANOVA followed by Student-Newmann-Keuls test. FIGS. 18C-18D show effects of NtB and lycopene combination on androgen-stimulated PSA production in human prostate cancer cells stimulated with testosterone (T) (FIG. 18C) or dihydrotestosterone (DHT) (FIG. 18D). Data are expressed as mean±SEM of ng/ml of PSA normalized by number of cells in each well determined by absorbance at 590 nm after crystal violet staining. Number of determinations are in parenthesis. * p<0.05 vs. control (without T or DHT), \ p<0.05 vs. T or DHT by one factor ANOVA followed by Student-Newmann-Keuls test.

FIGS. 19A-19B show influence of lycopene (LYC; 0.25 mg/ml) on relaxation induced by N. tangutorum Bobr extract (NtB) on norepinephrine (NE)-contracted human bladder neck (A) and prostate (B) strips from patients with BPH/LUTS. Data are expressed as mean±SEM of the reversion of NE-induced contraction. n indicates number of patients.

FIGS. 20A-20B show relaxation induced by lycopene (LYC) on norepinephrine (NE)-contracted human bladder neck (A) and prostate (B) strips from patients with BPH/LUTS. Panel A shows an example of concentration response curve to cumulative addition of lycopene vs. the effects of the vehicle (dimethylsulfoxide, DMSO) while panel B shows a tracing demonstrating the relaxant capacity (reduction of tension) of a single concentration of lycopene (0.25 mg/ml). N indicates number of patients.

FIGS. 21A-21B show effects of two-weeks daily oral administration of N. tangutorum Bobr extract (NtB; 30 mg/kg) plus lycopene (3 mg/kg) prostate hypertrophy and urodynamic parameters in rats with testosterone-induced BPH. Data were collected from seven rats treated with vehicle (tap water), five rats treated with NtB extract and three rats treated with NtB plus lycopene, and are expressed as mean±SEM. * p<0.05 vs. vehicle-treated rats by unpaired Student t-test. Graphs show the effects of NtB alone or in combination with lycopene on prostate weight/body weight ratio (FIG. 21A) and total bladder activity during infusion (area under the curve of intravesical pressure VP) (FIG. 21B).

FIG. 22 shows relaxation induced by N. tangutorum Bobr extract (NtB) on phenylephrine (PE)-contracted rat corpus cavernosum strips. Data are expressed as mean±SEM of the percentage of reversion of PE-induced contraction. N indicates the number of animals.

FIG. 23 shows a schematic diagram summarizing the results in the examples.

FIG. 24 shows an administration scheme according to one example of the present disclosure.

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications referred to in this application are incorporated by reference in their entireties for all purposes to the same extent as if each individual publication were individually incorporated by reference.

All headings are for the convenience of the reader and should not be used to limit the meaning of the text that follows the heading, unless so specified.

### Definitions

As used herein and in the appended claims, the singular forms “a,” “an,” and “the” include plural referents unless the context clearly dictates otherwise. For example, “a” or “an” means “at least one” or “one or more.” Thus, reference to “an ingredient” refers to one or more ingredients, and reference to “the method” includes reference to equivalent steps and methods disclosed herein and/or known to those skilled in the art, and so forth.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It is understood that aspects and embodiments of the disclosure described herein include “consisting” and/or “consisting essentially of” aspects and embodiments. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

As used herein, a “sample” can be a solution, a suspension, liquid, powder, a paste, aqueous, non-aqueous or any combination thereof.

The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” (API) is the pharmaceutically active substance in a drug.

The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

The term “pharmaceutically acceptable carrier” as used herein refers to an excipient, diluent, preservative, solubilizer, emulsifier, adjuvant, and/or vehicle. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy. 20^{th} ed., (Lippincott, Williams & Wilkins 2003). Except insofar as any conventional media or agent is incompatible with the active compound, such use in the compositions is contemplated.

As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. More specific embodiments are included in the Pharmaceutical Preparations and Methods of Administration section below. In some embodiments, the term “therapeutically effective amount” or “effective amount” refers to an amount of a therapeutic agent that when administered alone or in combination with an additional therapeutic agent to a cell, tissue, or subject is effective to prevent or ameliorate the disease or condition such as a hemolytic disease or condition, or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

As used herein, “preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition.

The term “combination” refers to either a fixed combination in one dosage unit form, or a kit of parts for the combined administration where a pharmaceutical composition, active ingredient, healthcare product, and/or food additive disclosed herein and a combination partner (e.g., another drug or extract as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently or sequentially with no specific time limits, wherein such administration provides therapeutically effective levels of the two moieties or compounds in the body of the patient. The latter also applies to cocktail therapy, e.g., the administration of three or more active ingredients.

As used herein, a subject in need refers to an animal, such as a human. In certain embodiments, a non-human mammal is also included. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

### Method of Extraction and Extracted Products

Nitraria tangutorum Bobr. belongs to family Nitrariaceae and is mainly distributed in northern Xinjiang area, China. It is also called “Desert Cherry” which the consumption of fruit nourishes stomach, spleen and lungs. Medicinally used to help digestion, nerve soothing, lactation, prevent neurasthenia, and promote blood circulation. It contains a wide variety of nutrients including vitamin C, polysaccharides, unsaturated fatty acids, proteins, amino acids, minerals (such as Zn, Cu, and Mn) and approximately 21 types of other trace elements. Nitrariaceae is a family of flowering plants in the order Sapindales. It comprises three genera, Nitraria, Peganum, and Tetradiclis, totaling aboutl19 species. The Nitraria genus includes species such as Nitraria billardierei DC. (known as Nitre Bush or Dillon Bush), Nitraria retusa (Forssk.) Asch., Nitraria schoberi L., and Nitraria sibirica Pall. Family Nitrariaceae used to be placed in family Zygophyllaceae. As such, Nitraria tangutorum Bobr. may relate to plant species within the Zygophyllaceae family as well.

In one aspect, provided herein is a method for preparing a Nitraria extract, for example an active Nitraria fruit extract with one or more health benefits for a human or animal In one aspect, the method disclosed herein overcomes one or more deficiencies of the art methods and/or maximally retain many, most, or all of the active components from the plant, such as a Nitraria fruit. In another aspect, provided herein is a method of using the Nitraria extract obtained by the method disclosed herein in the medicinal and health-care fields.

Although many examples herein refer to Nitraria tangutorum Bobr. and fruits thereof, it is to be understood that other parts of Nitraria tangutorum Bobr. may be used for extraction as described herein, such as leaves, roots, barks, stems, branches, flowers, and/or seeds of the plant. In addition, other plants and parts thereof can also be used for extraction as described herein. For example, fruits from other plants in family Nitrariaceae, or other plants in genus Nitraria, may be extracted using a method disclosed herein. In particular embodiments, fruits of Nitraria billardierei DC., Nitraria retusa (Forssk.) Asch., Nitraria schoberi L., and/or Nitraria sibirica Pall., either alone or mixed with one other or with Nitraria tangutorum Bobr. fruits, may be extracted using a method disclosed herein, and pharmaceutical compositions, active ingredients, healthcare products, and/or food additives can be obtained therefrom as described herein.

In another example, related plants in family Zygophyllaceae and fruits thereof may be extracted in order to obtain pharmaceutical compositions, active ingredients, healthcare products, and/or food additives as described herein. Zygophyllaceae is an established biological family and includes genera such as Fagonia, Guaciacum, Kallstroemia, Larrea, Peganum, Porlieria, and Tribulus. For example, plant extracts can be obtained from the leaves or stems of plants of the Larrea genus. Species within that genus include L. nitida, L. ameghinoi, L. divaricata, L. tridentata, and L. cuneifolia.

In any of the preceding embodiments, the various plants and parts thereof (either alone or in combination with one or more other plants) can be extracted using a method disclosed herein, and pharmaceutical compositions, active ingredients, healthcare products, and/or food additives can be obtained therefrom and used as described herein.

A. Supercritical Extraction

Supercritical Fluid Extraction (SFE), also referred to as supercritical extraction herein, is the process of separating one component (the extractant) from another (the matrix) using one or more supercritical fluids as the extracting solvent. Extraction is usually from a solid matrix, but can also be from liquids. SFE can be used as a sample preparation step for analytical purposes, or on a larger scale to either strip unwanted material from a product (e.g., decaffeination) or collect a desired product (e.g., essential oils). These essential oils can include limonene and other straight solvents. Carbon dioxide (CO₂) is a frequently used supercritical fluid, sometimes modified by co-solvents such as alcohols, ketones, ethers or esters. Extraction conditions for supercritical carbon dioxide are above the critical temperature of 31° C. and critical pressure of 74 bar. Addition of modifiers may slightly alter the critical temperature and/or the critical pressure.

The properties of a supercritical fluid can be altered by varying the pressure and temperature, allowing selective extraction. For example, volatile oils can be extracted from a plant with low pressures (100 bar), whereas liquid extraction would also remove lipids. Lipids can be removed using pure CO₂ at higher pressures, and then phospholipids can be removed by adding ethanol to the solvent. The same principle can be used to extract polyphenols and unsaturated fatty acids separately.

Extraction is a diffusion-based process, in which the solvent is required to diffuse into the matrix and the extracted material to diffuse out of the matrix into the solvent. Diffusivities are much faster in supercritical fluids than in liquids, and therefore extraction can occur faster. In addition, due to the lack of surface tension and negligible viscosities compared to liquids, the solvent can penetrate more into the matrix inaccessible to liquids. An extraction using an organic liquid may take several hours, whereas supercritical fluid extraction can be completed between about 10 minutes and about 60 minutes.

Carbon dioxide itself is non-polar, and has somewhat limited dissolving power. Therefore, particularly for polar solutes, the use of modifiers in addition to carbon dioxide increases the range of materials which can be extracted. Food grade modifiers such as ethanol can often be used, and can also help in the collection of the extracted material.

In one aspect, the present disclosure adopts the following technical solutions based on supercritical extraction.

In one embodiment, a method for preparing a plant (such as Nitraria fruit) extract is provided. In one aspect, disclosed herein is a method for obtaining an extract from Nitraria tangutorum Bobr., comprising: (1) extracting a sample of Nitraria tangutorum Bobr. fruit in a first supercritical fluid extraction device, to obtain an oil-like extract and a remaining sample; (2) mixing the remaining sample with an entrainer and extracting the mixture in a second supercritical fluid extraction device, to obtain a liquid phase sample; and (3) allowing evaporation from the liquid phase sample, and the resulting sample after evaporation comprises an extract comprising one or more ingredients from Nitraria tangutorum Bobr.

In some embodiments, the method comprises extracting a sample of Nitraria tangutorum Bobr. fruit in a first supercritical fluid extraction device, and an oil-like extract and a remaining sample are obtained as a result of this step. In any of the preceding embodiments, the method can further comprise mixing the remaining sample with an entrainer (also known as co-solvent) and extracting the mixture in a second supercritical fluid extraction device, and a liquid phase sample is obtained as a result of this step. In any of the preceding embodiments, the remaining sample in the mixing step can be a defatted remaining sample.

In one aspect, the method comprises obtaining a plant sample, such as a Nitraria fruit raw material. In any of the preceding embodiments, the method can further comprise obtaining the sample of Nitraria tangutorum Bobr. fruit before the extracting step. In any of the preceding embodiments, the method can further comprise cutting, shredding, mincing, or milling the sample of Nitraria tangutorum Bobr. fruit before the extracting step.

In one aspect, the method further comprises extracting, in a supercritical extraction equipment, the plant sample for between about 1 hour and about 3 hours (such as 2 hours) under a pressure of between about 20 MPa and about 35 MPa, at a temperature of between about 35° C. and about 55° C., and at a supercritical fluid flow rate between 0.5 L/min and about 3 L/min (such as 2 L/min), for example, between about 1 L/min and about 3 L/min (such as a CO₂ flow rate between about 1 L/min and about 2 L/min). In one other aspect, after the extraction, the method further comprises separating an oil-like extract from a defatted sample. In one aspect, the method further comprises fully infiltrating the defatted sample with an entrainer, and then extracting, in the supercritical extraction equipment, for between about 1 hour and about 3 hours, under a pressure of between about 20 MPa and about 35 MPa, at a temperature of between about 35° C. and about 55° C., at a CO₂ flow rate of about 1 L/min, and at an entrainer flow rate of between about 0.2 mL/min and about 1.0 mL/min, to obtain an extracted solution. In one aspect, the method further comprises drying the extracted solution to obtain a powder extract or a semi-solid extract.

In any of the preceding embodiments, the Nitraria fruit raw material can be a combination of one or more selected from the group consisting of a fresh Nitraria fruit, a dried Nitraria fruit, a Nitraria fruit pulp, and a Nitraria fruit seed. In any of the preceding embodiments, the sample ofNitraria tangutorum Bobr. fruit can comprise an intact fruit, flesh of the fruit, fruit pulp, a seed, a fresh fruit, a dried fruit, a chilled fruit, a frozen fruit, a preserved fruit, a milled fruit, a minced fruit, a crushed fruit, a granulated fruit, a powered fruit, or any combination thereof. In any of the preceding embodiments, the sample of Nitraria tangutorum Bobr. fruit can be a dried sample, a semi-wet sample, or a wet sample. In any of the preceding embodiments, the sample of Nitraria tangutorum Bobr. fruit can comprise about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 99.5%, or about 99.9% water by weight.

In any of the preceding embodiments, about 10 g of the Nitraria fruit raw material can be used for extraction. In any of the preceding embodiments, between about 10 g and about 500 kg (such as about 10 g, 200 kg, or 500 kg) of the sample of Nitraria tangutorum Bobr. fruit can be used for each extraction.

In any of the preceding embodiments, the supercritical fluid can comprise CO₂. In any of the preceding embodiments, the first and second supercritical fluid extraction devices can be the same or different. In any of the preceding embodiments, the entrainer can comprise an alcohol (e.g., ethanol) and/or water. The alcohol used herein can be any suitable alcohol, for example, an alcohol with up to about 10 carbon atoms (e.g., a C₅-alcohol or a C₁₀-alcohol) or any suitable combination thereof. In particular embodiments, an alcohol with no more than about 5 carbon atoms is used, for example, a C₁-alcohol, a C₂-alcohol, a C₃-alcohol, a C₄-alcohol, or a C₅-alcohol, or any combination thereof. In any of the preceding embodiments, the alcohol can be a primary alcohol, a secondary alcohol, or a tertiary alcohol. In some embodiments, the alcohol is a monohydric alcohol, such as methanol (CH₃OH), ethanol (C₂H₅OH), propan-2-ol (C₃H₇OH), butan-1-ol (C₄H₉OH), or pentan-1-ol (C₅H₁₁OH). In other embodiments, the alcohol is a polyhydric alcohol, such as ethane-1,2-diol [C₂H₄(OH)₂], propane-1,2-diol [C₃H₆(OH)₂], propane-1,2,3-triol [C₃H₅(OH)₃], butane-1,2,3,4-tetraol [C₄H₆(OH)₄], or pentane-1,2,3,4,5-pentol [C₅H₇(OH)₅]. In yet other embodiments, the alcohol is an unsaturated aliphatic alcohol, such as prop-2-ene-1-ol (C₃H₅OH) or prop-2-yn-1-ol (C₃H₃OH). In yet other embodiments, the alcohol is an alicyclic alcohol. In particular embodiments, the alcohol is a “green” material that does not cause pollution, such as ethanol, methanol, or the propyl alcohols.

In any of the preceding embodiments, the entrainer can be a mixture of ethanol and water. In one aspect, the entrainer comprises between about 35% (v/v) and about 95% (v/v) ethanol. In any of the preceding embodiments, the ratio between the volume of the entrainer and the weight of the remaining sample in the mixing step can be about (1 mL):(1 g). In one other aspect, the volume-to-mass ratio of the entrainer to the defatted sample is less than about (1 mL):(1 g), such as less than about (0.1 mL):(1 g), between about (0.1 mL):(1 g) and about (0.5 mL):(1 g), or between about (0.5 mL):(1 g) and about (1 mL):(1 g).

In any of the preceding embodiments, the remaining sample can be partially or fully infiltrated with the entrainer in the mixing step. In particular embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the entrainer has infiltrated the remaining sample. In particular embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the volume or mass of the remaining sample is infiltrated with the entrainer.

In any of the preceding embodiments, the defatted sample, after being infiltrated with the entrainer and prior to being extracted, can be statically soaked in supercritical CO₂ for between about 5 minutes and about 5 hours, for example, 10 min, 20 min, 30 min, 40 min, 50 min, or one hour. In particular embodiments, in the mixing step, the remaining sample, after being infiltrated with the entrainer and prior to the second supercritical fluid extraction, can be statically soaked in the supercritical fluid (e.g., CO₂), e.g., for about 30 minutes.

In any of the preceding embodiments, between about 5 g and about 500 kg (such as about 5 g, 100 kg, 250 kg, or 500 kg) of the remaining sample in the mixing step can be used for the second supercritical fluid extraction. In any of the preceding embodiments, about 5 g of the defatted sample can be used for each subsequent extraction.

In any of the preceding embodiments, the method can further comprise purifying or isolating the extract and/or the ingredient from the resulting sample after evaporation. In any of the preceding embodiments, the method can further comprise rotatable evaporating the liquid phase sample before drying, at a temperature between about 35° C. and about 55° C. (e.g., at about 50° C.), e.g., in order to remove a solvent in the liquid phase sample. In any of the preceding embodiments, the method can further comprise rotatable evaporating the liquid phase sample before drying, under a pressure of about −0.09 MPa, e.g., in order to remove a solvent in the liquid phase sample. In any of the preceding embodiments, the extracted solution, before being dried, can be rotatable evaporated at a temperature of between about 40° C. and about 50° C., and under a vacuum pressure of about −0.09 MPa, in order to remove the solvent.

In any of the preceding embodiments, the method can further comprise drying the resulting sample after evaporation. In any of the preceding embodiments, the drying can be carried out at a temperature of about −50° C. and/or under a pressure of about 5 Pa. In any of the preceding embodiments, the drying step can comprise freeze-drying (lyophilization) and/or spray-drying. In any of the preceding embodiments, the drying can be performed at a freeze-drying temperature of about −50° C. and under an absolute pressure of about 5 Pa. In some embodiments, the freeze-drying temperature is about −100° C., −90° C., −80° C., −70° C., −60° C., −50° C., −40° C., −30° C., −20° C., or about −10° C. In some embodiments, the freeze-drying pressure is about 1 Pa, about 2 Pa, about 3 Pa, about 4 Pa, about 5 Pa, about 6 Pa, about 7 Pa, about 8 Pa, about 9 Pa, or about 10 Pa.

Other suitable drying methods can be used, for example, a next-generation drying technology may be used for drying the pharmaceutical composition, active ingredient, healthcare product, and/or food additives herein. For a review of available drying methods, see Walters et al., Next generation drying technologies for pharmaceutical applications, 2014, J Pharm Sci. 103(9):2673-95.

In any of the preceding embodiments, the method can further comprise obtaining a powder extract or a semi-solid extract comprising one or more desired ingredients.

B. Solvent Extraction

In one aspect, disclosed herein is a method for obtaining an extract from Nitraria tangutorum Bobr. In one aspect, the method comprises mixing a sample of Nitraria tangutorum Bobr. fruit with an alcohol (e.g., ethanol) solution, e.g., from about 30% (v/v) to about 95% (v/v), such as about 65% (v/v). The alcohol used herein can be any suitable alcohol, for example, an alcohol with up to about 10 carbon atoms (e.g., a C₅-alcohol or a C₁₀-alcohol) or any suitable combination thereof. In particular embodiments, an alcohol with no more than about 5 carbon atoms is used, for example, a C₁-alcohol, a C₂-alcohol, a C₃-alcohol, a C₄-alcohol, or a C₅-alcohol, or any combination thereof. In any of the preceding embodiments, the alcohol can be a primary alcohol, a secondary alcohol, or a tertiary alcohol. In some embodiments, the alcohol is a monohydric alcohol, such as methanol (CH₃OH), ethanol (C₂H₅OH), propan-2-ol (C₃H₇OH), butan-1-ol (C₄H₉OH), or pentan-1-ol (C₅H₁₁OH). In other embodiments, the alcohol is a polyhydric alcohol, such as ethane-1,2-diol [C₂H₄(OH)₂], propane-1,2-diol [C₃H₆(OH)₂], propane-1,2,3-triol [C₃H₅(OH)₃], butane-1,2,3,4-tetraol [C₄H₆(OH)₄], or pentane-1,2,3,4,5-pentol [C₅H₇(OH)₅]. In yet other embodiments, the alcohol is an unsaturated aliphatic alcohol, such as prop-2-ene-1-ol (C₃H₅OH) or prop-2-yn-1-ol (C₃H₃OH). In yet other embodiments, the alcohol is an alicyclic alcohol. In particular embodiments, the alcohol is a “green” material that does not cause pollution, such as ethanol, methanol, or the propyl alcohols.

In another aspect, the method comprises incubating the mixture under a temperature between about 10° C. and about 60° C., such as between about room temperature (e.g., about 18° C.) and about 55° C. In yet another aspect, the method comprises obtaining a liquid phase sample from the mixture. In one aspect, the method comprises filtering the liquid phase sample, removing the alcohol from the liquid phase sample, and/or concentrating the liquid phase sample. In any of the preceding embodiments, the remaining sample can comprise a solid, semi-solid, and/or liquid matter. In one other aspect, the method comprises allowing evaporation of the alcohol and/or water from the liquid phase sample. In any of the preceding embodiments, the resulting sample after evaporation can comprise an extract comprising one or more ingredients from Nitraria tangutorum Bobr.

In another aspect, provided herein is a method for preparing a plant (such as Nitraria fruit) extract, such as an active Nitraria fruit extract. In one aspect, the method comprises obtaining a raw material from a plant, such as a Nitraria fruit raw material. In one aspect, the method further comprises, in a extraction equipment, performing an extraction by mixing and/or stirring the Nitraria fruit raw material with ˜65% (v/v) ethanol at a temperature of between about 18° C. and about 55° C. In one aspect, the method further comprises separating a liquid extract from a solid sample after the extraction. In one aspect, the method further comprises purifying and/or drying the liquid extract to obtain a powder extract therefrom.

In any of the preceding embodiments, the Nitraria fruit raw material can be a combination of one or more selected from the group consisting of a fresh Nitraria fruit, a dried Nitraria fruit, a Nitraria fruit pulp, and a Nitraria fruit seed. In any of the preceding embodiments, the sample of Nitraria tangutorum Bobr. fruit can comprise an intact fruit, flesh of the fruit, fruit pulp, a seed, a fresh fruit, a dried fruit, a chilled fruit, a frozen fruit, a preserved fruit, a milled fruit, a minced fruit, a crushed fruit, a granulated fruit, a powered fruit, or any combination thereof. In any of the preceding embodiments, the sample of Nitraria tangutorum Bobr. fruit can be a dried sample, a semi-wet sample, or a wet sample. In any of the preceding embodiments, the sample of Nitraria tangutorum Bobr. fruit can comprise about 0.1%, about 0.5%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 99.5%, or about 99.9% water by weight.

In any of the preceding embodiments, the method can further comprise obtaining the sample of Nitraria tangutorum Bobr. fruit before the mixing step. In any of the preceding embodiments, the Nitraria fruit raw material can be smashed or chopped before being extracted. In any of the preceding embodiments, the method can further comprise cutting, shredding, mincing, or milling the sample of Nitraria tangutorum Bobr. fruit before the mixing step.

In any of the preceding embodiments, in the mixing step, the ratio between the sample weight and the alcohol solution volume can be between about (1 g):(3 mL) and about (1 g):(10 mL), optionally between about (1 g):(3 mL) and about (1 g):(5 mL). In any of the preceding embodiments, the mass-to-volume ratio of the plant (e.g., Nitraria fruit) raw material to the alcohol (e.g., 65% ethanol) can be between about (1 g):(3 mL) and about (1 g):(10 mL), for example, about (1 g):(4 mL), about (1 g):(5 mL), about (1 g):(6 mL), about (1 g):(7 mL), about (1 g):(8 mL), or about (1 g):(9 mL).

In any of the preceding embodiments, the stirring extraction can be performed for between about 1 hour and about 2 hours.

In any of the preceding embodiments, the stirring extraction can be repeated for once or twice, or three or four times, or more.

In any of the preceding embodiments, the incubating step can be carried out while stirring the mixture, e.g., for extraction of the ingredient into the liquid phase sample. In any of the preceding embodiments, a pressure treatment and/or an ultrasonic vibration treatment can also be performed during the stirring extraction.

In any of the preceding embodiments, the method can further comprise a pressure treatment and/or ultra-sonication during the mixing, incubating, and/or obtaining step. In one aspect, the pressure is between about 5 MPa and about 150 MPa, e.g., 30 MPa or 50 MPa. In any of the preceding embodiments, the pressure of the pressure treatment can be between about 30 MPa and about 300 MPa, for example, 100 MPa or 150 MPa. In any of the preceding embodiments, the pressure of the pressure treatment can be between about 5 MPa and about 150 MPa. In any of the preceding embodiments, the power of the ultrasonic vibration can between about 100 W and about 10,000 W, such as about 600 W.

In any of the preceding embodiments, in the obtaining step, the liquid phase sample can be extracted from the mixture. In any of the preceding embodiments, the remaining sample can comprise mostly solid matter. For example, at least about 50%, at least about 55%,at least about 60%, at least about 65%, at least about 70%, at least about 75%, at least about 80%, at least about 85%, at least about 90%, at least about 95%, or at least about 99% of the mass of the remaining sample is solid matter.

In any of the preceding embodiments, the method can further comprise purifying or isolating the extract and/or the ingredient from the resulting sample after evaporation.

In any of the preceding embodiments, the method can further comprise drying the resulting sample after evaporation. In any of the preceding embodiments, a powder extract comprising the ingredient can be obtained.

In any of the preceding embodiments, after obtaining the liquid phase sample, the method can further comprise: (a) obtaining from the mixture a remaining sample which comprises mostly solid matter, and the mixture is a first mixture; (b) mixing the remaining sample with an alcohol (e.g., ethanol) solution, e.g., from about 30% (v/v) to about 95% (v/v), such as about 65% (v/v), and optionally the ratio between the weight of the remaining sample and the alcohol solution volume is between about (1 g):(3 mL) and about (1 g):(10 mL) and optionally between about (1 g):(3 mL) and about (1 g):(5 mL), to obtain a second mixture; (c) incubating the second mixture under a temperature between about room temperature (e.g., about 18° C.) and about 55° C.; (d) obtaining a second liquid phase sample from the second mixture, and the liquid phase sample from the first mixture is a first liquid phase sample; (e) combining the first and second liquid phase samples, and optionally filtering the combined liquid phase sample; and (f) allowing evaporation from the combined liquid phase sample, and the resulting sample after evaporation comprises the extract comprising one or more ingredients from Nitraria tangutorum Bobr.

In one embodiment, steps (a)-(d) are repeated one, two, or more times before step (e). In another embodiment, steps (a)-(e) are repeated one, two, or more times before step (f).

In any of the preceding embodiments, the purification of the liquid extract can comprises at least one of the following: rotatably evaporating at a temperature of about 50° C. and/or under a reduced pressure, such as between about −5 MPa and about 5 MPa, in order to remove a solvent in the liquid extract; performing chromatography, for example, by a macro-porous resin adsorption column, to remove one or more sugars in the liquid extract; and performing chromatography, for example, by a macro-porous resin adsorption column, to increase the anthocyanin and/or polyphenol content in the liquid extract.

In any of the preceding embodiments, the method can further comprise rotatably evaporating the liquid phase sample at a temperature of between about 20° C. and about 70° C. (such as about 55° C.) and/or a reduced pressure (such as between about −5 MPa and about 5 MPa), e.g., in order to remove a solvent in the liquid phase sample.

In any of the preceding embodiments, the method can further comprise passing the liquid phase sample through a first chromatography column. In any of the preceding embodiments, the first chromatography column can comprise a macroporous resin adsorption column. In any of the preceding embodiments, passing the liquid phase sample through the first chromatography column can remove one or more sugars or polysaccharides from the liquid phase sample.

In any of the preceding embodiments, the method can further comprise passing the liquid phase sample through a second chromatography column. In any of the preceding embodiments, the second chromatography column can comprise a macroporous resin adsorption column. In any of the preceding embodiments, passing the liquid phase sample through the second chromatography column can increase the anthocyanin and/or polyphenol concentration in the liquid phase sample.

In any of the preceding embodiments, the first and second chromatography columns can be the same or different.

In any of the preceding embodiments, before the chromatography, a part or substantially all of the alcohol can be removed (e.g., by drying or rotatably evaporating the alcohol) from the liquid phase sample, which is then re-dissolved in water. As used herein, substantially all of the alcohol can include about 60%, about 65%, about 70%, about 75%, about 80%, about 85%, about 90%, about 95%, about 99%, or about 99.9% of the alcohol.

In any of the preceding embodiments, the chromatography can comprise eluting the column, e.g., with an alcohol solution of about 50% (v/v) to about 100% (v/v), such as about 95% (v/v).

In any of the preceding embodiments, the allowing step can comprise freeze-drying (lyophilization) and/or spray-drying the resulting sample to obtain the extract.

C. Compositions, Formulations, Healthcare Products, Food Additives, and Methods of Use

In another aspect, disclosed herein is a pharmaceutical composition comprising one or more selected from the group consisting of the oil-like extract, the defatted sample, the extracted solution, and the powder extract obtained from the plant sample, such as the Nitraria fruit sample.

In another aspect, disclosed herein is a pharmaceutical composition comprising one or more selected from the group consisting of the liquid extract, the solid sample, and the powder extract obtained from the plant sample, such as the Nitraria fruit sample.

In any of the preceding embodiments, the one or more ingredients can comprise one or more anthocyanins and/or one or more polyphenols.

Anthocyanins (also anthocyans) are water-soluble vacuolar pigments that may appear red, purple, or blue depending on the pH. They belong to a parent class of molecules called flavonoids synthesized via the phenylpropanoid pathway; they are odorless but flavorful, contributing to taste as a moderately astringent sensation. Anthocyanins occur in all tissues of higher plants, including leaves, stems, roots, flowers, and fruits. Anthocyanins are derived from anthocyanidins by adding sugars. Anthocyanin plays a key role in many plant species as an antioxidant. The production of reactive oxygen species can be caused by abiotic stresses such as over exposure to ultraviolet light, exposure to lower than desirable temperatures, and presumably many more. Reactive oxygen species are necessary for cell signaling during regular growth and development, but an over accumulation can lead to harmful oxidative stress. Anthocyanin-rich plants have been shown to contain a healthier level of reactive oxygen species when under cold stress leading to a significantly lower rate of cell death in leaves. Anthocyanins are considered secondary metabolites as a food additive.

Polyphenols are phytochemicals which are found abundantly in natural plant food sources that have antioxidant properties. There are over 8,000 identified polyphenols found in foods such as tea, wine, chocolates, fruits, vegetables, and extra virgin olive oil. Polyphenols can include flavonoids (such as flavones, flavonols, flavanones, isoflavones, anthocyanidins, chalcones, and catechins), stilbenes, lignans, and phenolic acids (such as hydroxybenzoic acids and hydroxycinnamic acids). Polyphenols give fruits, berries, and vegetables their vibrant colors, and contribute to the bitterness, astringency, flavor, aroma, and oxidative stability of the food. In the plant, they protect against ultraviolet radiation, pathogens, oxidative damage, and harsh climatic conditions. In the human body, polyphenols have diverse biological properties, such as fighting cancer cells and inhibiting angiogenesis, protecting skin against ultraviolet radiation, fighting free radicals, and reducing the appearance of aging, promoting brain health, and protecting against dementia, reducing inflammation, supporting normal blood sugar levels, protecting the cardiovascular system, and promoting normal blood pressure.

In yet another aspect, provided herein is a liquid phase sample, a remaining sample, a resulting sample, an extract, and/or one or more ingredients from Nitraria tangutorum Bobr. produced by the method according to any one of the preceding embodiments.

In still another aspect, provided herein is a liquid phase sample, a remaining sample, a resulting sample, an extract, and/or one or more ingredients from Nitraria tangutorum Bobr. produced by the method according to any one of the preceding embodiments, for use in the treatment and/or prevention of a condition or disease in a subject in need thereof.

In one aspect, provided herein is use of the liquid phase sample, the remaining sample, the resulting sample, the extract, and/or the one or more ingredients from Nitraria tangutorum Bobr. of any one of the preceding embodiments, in the manufacture of a medicament for treating and/or preventing a condition or disease in a subject in need thereof.

In another aspect, provided herein is an oil-like extract, a remaining sample, a liquid phase sample, a resulting sample, an extract, and/or one or more ingredients from Nitraria tangutorum Bobr. produced by the method according to any one of the preceding embodiments. Also provided herein is the oil-like extract, the remaining sample, the liquid phase sample, the resulting sample, the extract, and/or the one or more ingredients from Nitraria tangutorum Bobr. for use in the treatment and/or prevention of a condition or disease in a subject in need thereof.

In one aspect, provided herein is use of the oil-like extract, the remaining sample, the liquid phase sample, the resulting sample, the extract, and/or the one or more ingredients from Nitraria tangutorum Bobr. of any one of the preceding embodiments, in the manufacture of a medicament for treating and/or preventing a condition or disease in a subject in need thereof.

In any of the preceding embodiments, the pharmaceutical composition can be used for preventing and/or treating a condition or a disease, and/or for providing a health benefit, for a mammal such as a human. In one aspect, the condition or disease is a lower urinary tract symptom, for example, due to a benign prostatic hypertrophy.

In any of the preceding embodiments, the pharmaceutical composition can further comprise a lycopene, which can be natural or synthetic. The pharmaceutical composition can be used in a mixture with a lycopene extract or composition. Alternatively, the pharmaceutical composition can be administered to a subject in combination with a lycopene extract or composition. The compositions can be simultaneously or sequentially administered. In any of the preceding embodiments, the pharmaceutical composition can further comprise saw palmetto or an extract thereof, pumpkin seed or an extract thereof (such as a protein extract), Selenium (Se), Lyceum ruthenicum or an extract thereof, black tomato or an extract thereof, lentinan, Pleurotus ostreatus polysaccharide, agaric polysaccharide, Flammulina velutipes polysaccharide, spirulina or an extract thereof, lutein, zeaxanthin, and/or astaxanthin.

Lycopene from the neo-Latin lycopersicum, the tomato species, is a bright red carotene and carotenoid pigment and phytochemical found in tomatoes and other red fruits and vegetables, such as red carrots, watermelons, gac (Momordica cochinchinensis), and papayas. Although lycopene is chemically a carotene, it generally has no vitamin A activity. Foods that are not red may also contain lycopene, such as asparagus and parsley. In one aspect, provided herein is a composition, extract, or plant part comprising lycopene, such as a red fruit extract or a vegetable extract, including a red carrot extract, a watermelon extract, a gac extract, and a papaya extract, and a tomato extract.

In plants, algae, and other photosynthetic organisms, lycopene is an important intermediate in the biosynthesis of many carotenoids, including beta-carotene, which is responsible for yellow, orange, or red pigmentation, photosynthesis, and photoprotection. Like all carotenoids, lycopene is a polyunsaturated hydrocarbon, i.e. an unsubstituted alkene. Structurally, lycopene is a tetraterpene and assembled from eight isoprene units that are composed entirely of carbon and hydrogen. It is insoluble in water. Lycopene's eleven conjugated double bonds give its deep red color and its antioxidant activity in vitro. Owing to the strong color, lycopene is a useful food coloring (registered as E160d) and is approved for usage in the USA, Australia and New Zealand (registered as 160d) and the EU.

In yet another aspect, provided herein is a pharmaceutical composition comprising one or more selected from the group consisting of the oil-like extract, the defatted sample, the extracted solution, and the powder extract obtained by the supercritical extraction method; and the liquid extract, the solid sample, and the powder extract obtained by the solvent extraction method. For example, the pharmaceutical composition may comprise the oil-like extract obtained by the supercritical extraction method, and the powder extract obtained by the solvent extraction method. Any suitable combination of the various components is within the present disclosure.

In any of the preceding embodiments, the pharmaceutical composition can be used for preventing and/or treating a condition or a disease, and/or for providing a health benefit, for a mammal such as a human. In one aspect, the pharmaceutical composition is used for preventing and/or treating a degenerative disease, such as macular degeneration, including age-related macular degeneration. In another aspect, the pharmaceutical composition is used for preventing and/or treating an eye disease, a fundus lesion, a cancer, a cardiovascular disease, a vascular disease, a neural disease, an immune disorder, an infection, and/or an inflammation. In another aspect, the pharmaceutical composition is used for preventing and/or treating a condition or disease associated with radiation of radioactive rays or radiation of ultraviolet rays.

In one aspect, provided herein is a pharmaceutical composition comprising one or more selected from the group consisting of the oil-like extract, the defatted sample, the extracted solution and the powder extract obtained by the supercritical extraction method of extracting a Nitraria fruit sample, and/or comprising one or more selected from the group consisting of the liquid extract, the solid sample, and the powder extract obtained by the solvent extraction method of extracting a Nitraria fruit sample.

The present method can be used for any suitable purposes or applications. For example, the present method can be used for treating and/or preventing a disease or condition that is selected from the group consisting of an infectious disease, a parasitic disease, a neoplasm, a cancer, a disease of the blood and blood-forming organs, a disorder involving the immune mechanism, endocrine, nutritional and metabolic diseases, a mental and behavioral disorder, a disease of the nervous system, a disease of the eye, a disease of the ear and mastoid process, a disease of the circulatory system, a disease of the respiratory system, a disease of the digestive system, a disease of the skin and subcutaneous tissue, a disease of the musculoskeletal system and connective tissue, a disease of the genitourinary system, pregnancy, childbirth and the puerperium, a condition originating in the perinatal period, a congenital malformation, a deformation, a chromosomal abnormality, an injury, a poisoning, a consequence of external causes, and an external cause of morbidity and mortality.

In any of the preceding embodiments, the pharmaceutical composition can be used for regulating and restoring an immune function. In any of the preceding embodiments, the pharmaceutical composition can be used for preventing and/or treating a cancer or a cancer syndrome. In any of the preceding embodiments, the pharmaceutical composition can be for improving a vascular function, blood circulation, a cerebral function, and/or an immune function, and/or for preventing and/or alleviating a symptom and/or consequence of erectile dysfunction, hypertension, arteriosclerosis, thrombosis, fatigue, cerebral apoplexy, and/or stroke. In any of the preceding embodiments, the pharmaceutical composition can be for anti-thrombosis.

In any of the preceding embodiments, the pharmaceutical composition can be for alleviating a side effect of a therapy, such as treatment with an anticancer agent.

In any of the preceding embodiments, the pharmaceutical composition can be prepared in a form selected from the group consisting of a liquid, a powder a tablet, a granule, a pill, a capsule (e.g., a hard capsule or a soft capsule), an oral cream, a paste, a decoction, a syrup, a wine, a distillate, and any combination thereof. The pharmaceutical compositions comprising the active ingredients, products, and/or food additives described herein may further comprise one or more excipients such as pharmaceutically acceptable excipients. A pharmaceutically acceptable excipient is a substance that is non-toxic and otherwise biologically suitable for administration to a subject. Such excipients facilitate administration of the active ingredient(s) are compatible with the active ingredient(s). Examples of pharmaceutically acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the various embodiments are sterile compositions. Pharmaceutical compositions may be prepared using compounding techniques known or that become available to those skilled in the art.

The pharmaceutical compositions, active ingredients, products, and/or food additives described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms.

In any of the preceding embodiments, the pharmaceutical compositions, active ingredients, products, and/or food additives can be in a dosage form for oral, gastrointestinal, topical, mucosal, intravenous, intradermal, subcutaneous, or intramuscular administration. In some embodiments, the pharmaceutical compositions, active ingredients, products, and/or food additives may be administered by a suitable route of delivery, such as oral, parenteral, rectal, nasal, topical, or ocular routes, or by inhalation. In some embodiments, the compositions are formulated for intravenous or oral administration.

For oral administration, the pharmaceutical compositions, active ingredients, products, and/or food additives described herein may be provided in a solid form, such as a tablet or capsule, or as a solution, emulsion, or suspension. To prepare the oral compositions, the pharmaceutical compositions, active ingredients, products, and/or food additives described herein, alone or in combination with other active ingredient(s), may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with an oil, such as peanut oil or olive oil, liquid paraffin, bee wax, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

In another aspect, disclosed herein is a method of treating and/or preventing a condition and/or disease in a subject in need thereof, comprising administering to the subject a pharmaceutically effective dose of: (i) the liquid phase sample, the remaining sample, the resulting sample, the extract, and/or the one or more ingredients from Nitraria tangutorum Bobr. of any of the preceding embodiments; (ii) the oil-like extract, the remaining sample, the liquid phase sample, the resulting sample, the extract, and/or the one or more ingredients from Nitraria tangutorum Bobr. of any of the preceding embodiments; and/or (iii) the pharmaceutical composition according to any of the preceding embodiments.

In any of the preceding embodiments, the method can be used in combination with another therapy or regimen for treating and/or preventing the condition and/or disease.

In any of the preceding embodiments, the method can be used before, during, and/or after the other therapy or regimen, or in an alternating fashion with the other therapy or regimen.

In yet another aspect, provided herein is a health-care preparation comprising one or more selected from the group consisting of the oil-like extract, the defatted sample, the extracted solution and the powder extract obtained by the supercritical extraction method of extracting a Nitraria fruit sample, and/or comprising one or more selected from the group consisting of the liquid extract, the solid sample, and the powder extract obtained by the solvent extraction method of extracting a Nitraria fruit sample. In yet another aspect, provided herein is a health-care preparation comprising one or more selected from the group consisting of the oil-like extract, the defatted sample, the extracted solution and the powder extract obtained by the supercritical extraction method of extracting a Nitraria fruit sample. In still another aspect, provided herein is a health-care preparation comprising one or more selected from the group consisting of the liquid extract, the solid sample, and the powder extract obtained by the solvent extraction method of extracting a Nitraria fruit sample.

In any of the preceding embodiments, the health-care preparation can further comprise one or more excipients, for example, to form a dosage form for gastrointestinal administration.

In any of the preceding embodiments, the dosage form for gastrointestinal administration can be in any of the following forms: a powder, granule, pill, tablet, capsule, oral cream, paste, decoction, mixture, syrup, wine, distillate, and any suitable combination thereof.

In certain aspects, compared with the art methods, the present disclosure has the following beneficial effects.

(1) In one aspect of the presently disclosed method for preparing active Nitraria fruit extracts, the supercritical extraction technology is used and the extraction is performed under mild conditions. Fat extracts (oil-like extracts) can be fully obtained from different parts of a Nitraria fruit, and the defatted sample can be extracted again to obtain other active components. Therefore, the overall extraction efficiency of the Nitraria fruit is higher in the present method, as compared to the art methods.

(2) In one aspect of the presently disclosed method for preparing active Nitraria fruit extracts, an alcohol such as ethanol is used as a solvent for extraction and the extraction is performed under mild conditions. In addition, the yield of the extracts is high and thus is suitable for industrial production.

(3) In one aspect of the presently disclosed method for preparing active Nitraria fruit extracts, whether comprising the supercritical extraction technology and/or the solvent extraction method (e.g., with ethanol), no complex pre-treatment of the Nitraria fruit raw material is required before the extraction. Thus, the yield of active components obtained by extraction is high. The obtained components are rich in a variety of good quality substances that are beneficial to human health.

(4) The pharmaceutical composition containing the active Nitraria fruit extracts herein can contain one or more of the extracted products, which may be selected for use according to the specific indication to be prevented and/or treated. Thus, this is advantageous for the development of clinical use of the Nitraria fruit.

(5) The pharmaceutical composition containing the active Nitraria fruit extracts herein can further contain active components from other sources, such as a synthesized component or one isolated from another plant, which may be selected for use in a combination therapy or treatment according to the specific indication to be prevented and/or treated. This way, the active Nitraria fruit extracts can be combined with many other compositions in order to achieve better effect.

(6) The health-care preparation containing the active Nitraria fruit components in the present disclosure can contain one or more of the extracted products according to the specific indications to be prevented and/or treated. This is advantageous for the application and development of health-care preparations.

(7) For the health-care preparation containing the active Nitraria fruit components in the present disclosure, an appropriate dosage form can be selected according to the target population and the properties of the extracted Nitraria fruit products. This is advantageous to enrich the categories of Nitraria fruit health-care products.

Additional embodiments are provided below to further illustrate the present disclosure.

### Embodiment 1

A method for preparing an active Nitraria fruit extract, characterized in that comprising the following steps of:

obtaining a Nitraria fruit raw material;

extracting, in a supercritical extraction equipment, the Nitraria fruit raw material for 2-3 hours under a pressure of 20-35 MPa, at a temperature of 35-55° C. and at CO₂ flow rate of 1-2 L/min;

separating an oil-like extract from defatted residues obtained by the extraction;

fully infiltrating the defatted residues with an entrainer, and then extracting, in the supercritical extraction equipment, for 1-3 h under a pressure of 5-150 MPa (such as 20-35 MPa), at a temperature of 35-55° C., and at CO₂ flow rate of 1 L/min and the entrainer flow rate of 0.2-1.0 mL/min, to obtain an extracted solution; and

drying the extracted solution to obtain a powder extract or semi-solid extract.

### Embodiment 2

The method for preparing an active Nitraria fruit extract according to Embodiment 1, characterized in that the Nitraria fruit raw material is a combination of one or more selected from the group consisting of fresh Nitraria fruit, dried Nitraria fruit, Nitraria fruit pulp, and Nitraria fruit seed.

### Embodiment 3

The method for preparing an active Nitraria fruit extract according to Embodiment 1, characterized in that 10 g of the Nitraria fruit raw material is selected for extraction for each batch.

### Embodiment 4

The method for preparing an active Nitraria fruit extract according to Embodiment 1, characterized in that the entrainer is a mixture of ethanol and water.

### Embodiment 5

The method for preparing an active Nitraria fruit extract according to Embodiment 4, characterized in that the entrainer comprises between about 35% and about 95% ethanol.

### Embodiment 6

The method for preparing an active Nitraria fruit extract according to Embodiment 5, characterized in that the volume-to-mass ratio of the entrainer to the defatted residues is about (1 mL):(1 g).

### Embodiment 7

The method for preparing an active Nitraria fruit extract according to Embodiment 1, characterized in that the defatted residues, after being infiltrated with the entrainer and prior to being extracted, are statically soaked in supercritical CO₂ for about 30 min.

### Embodiment 8

The method for preparing an active Nitraria fruit extract according to Embodiment 1, characterized in that 5 g of the defatted residues is selected for extraction every time.

### Embodiment 9

The method for preparing an active Nitraria fruit extract according to Embodiment 1, characterized in that the extracted solution, before being dried, is rotatably evaporated at a temperature of 40-50° C. and under a vacuum pressure of −0.09 MPa, in order to remove a solvent.

### Embodiment 10

The method for preparing an active Nitraria fruit extract according to Embodiment 9, characterized in that the drying is performed at a freeze-drying temperature of −50° C. and under an absolute pressure of 5 Pa.

### Embodiment 11

A method for preparing an active Nitraria fruit extract, characterized in that comprising the following steps of:

obtaining a Nitraria fruit raw material;

in a extraction equipment, performing stirring extraction with the Nitraria fruit raw material and 65% ethanol at a temperature of 18-55° C.;

separating a liquid extract from solid residues obtained by the extraction; and

purifying and drying the liquid extract to obtain a powder extract.

### Embodiment 12

The method for preparing an active Nitraria fruit extract according to Embodiment 11, characterized in that the Nitraria fruit raw material is a combination of one or more selected from the group consisting of fresh Nitraria fruit, dried Nitraria fruit, Nitraria fruit pulp and Nitraria fruit seed.

### Embodiment 13

The method for preparing an active Nitraria fruit extract according to Embodiment 12, characterized in that the Nitraria fruit raw material is smashed or chopped before being extracted.

### Embodiment 14

The method for preparing an active Nitraria fruit extract according to Embodiment 11, characterized in that the mass-to-volume ratio of the Nitraria fruit raw material to the 65% ethanol is 1:3-10.

### Embodiment 15

The method for preparing an active Nitraria fruit extract according to Embodiment 14, characterized in that the stirring extraction is performed for 1-2 h.

### Embodiment 16

The method for preparing an active Nitraria fruit extract according to Embodiment 15, characterized in that the stirring extraction is repeated for once or twice.

### Embodiment 17

The method for preparing an active Nitraria fruit extract according to Embodiment 15, characterized in that pressure treatment or ultrasonic vibration treatment is also performed during the stirring extraction.

### Embodiment 18

The method for preparing an active Nitraria fruit extract according to Embodiment 17, characterized in that pressurized pressure of the pressure treatment is any one of 5-150 MPa; and the power of the ultrasonic vibration treatment is 600 W.

### Embodiment 19

The method for preparing an active Nitraria fruit extract according to Embodiment 11, characterized in that the purification of the liquid extract comprises at least one of the following:

rotatable evaporating at a temperature of 50° C. and under a reduced pressure, in order to remove a solvent in the liquid extract;

performing chromatography by a macro-porous resin adsorption column to remove sugar in the liquid extract; and

performing chromatography by a macro-porous resin adsorption column to increase anthocyanins and polyphenols in the liquid extract.

### Embodiment 20

The method for preparing an active Nitraria fruit extract according to Embodiment 11, characterized in that the drying is realized by freeze-drying or spray-drying.

### Embodiment 21

A pharmaceutical composition, characterized in that containing one or more selected from the group consisting of the oil-like extract, the defatted residues, the extracted solution and the powder extract obtained by the method for preparing an active Nitraria fruit extract according to any one of Embodiments 1-10, and/or containing one or more selected from the group consisting of the liquid extract, the solid residues and the powder extract obtained by the method for preparing an active Nitraria fruit extract according to any one of Embodiments 11-20, for preventing and/or treating lower urinary tract symptoms due to benign prostatic hypertrophy.

### Embodiment 22

The pharmaceutical composition according to Embodiment 21, characterized in that the pharmaceutical composition further comprises an extract containing lycopene.

### Embodiment 23

A pharmaceutical composition, characterized in that containing one or more selected from the group consisting of the oil-like extract, the defatted residues, the extracted solution and the powder extract obtained by the method for preparing an active Nitraria fruit extract according to any one of Embodiments 1-10, and/or containing one or more selected from the group consisting of the liquid extract, the solid residues and the powder extract obtained by the method for preparing an active Nitraria fruit extract according to any one of Embodiments 11-20, for preventing and/or treating macular degeneration.

### Embodiment 24

The pharmaceutical composition according to Embodiment 23, characterized in that the macular degeneration is associated with radiation of radioactive rays or radiation of ultraviolet rays, or the macular degeneration is age-related macular degeneration.

### Embodiment 25

A pharmaceutical composition, characterized in that containing one or more selected from the group consisting of the oil-like extract, the defatted residues, the extracted solution and the powder extract obtained by the method for preparing an active Nitraria fruit extract according to any one of Embodiments 1-10, and/or containing one or more selected from the group consisting of the liquid extract, the solid residues and the powder extract obtained by the method for preparing an active Nitraria fruit extract according to any one of Embodiments 11-20, for regulating and restoring the immune function.

### Embodiment 26

The pharmaceutical composition according to Embodiment 25, characterized in that the pharmaceutical composition further comprises pharmaceutical components for preventing and/or treating cancers.

### Embodiment 27

A health-care preparation, characterized in that containing one or more selected from the group consisting of the oil-like extract, the defatted residues, the extracted solution and the powder extract obtained by the method for preparing an active Nitraria fruit extract according to any one of Embodiments 1-10, and/or containing one or more selected from the group consisting of the liquid extract, the solid residues and the powder extract obtained by the method for preparing an active Nitraria fruit extract according to any one of Embodiments 11-20.

### Embodiment 28

The health-care preparation according to Embodiment 27, characterized in that further comprising excipients to form a dosage form for gastrointestinal administration.

### Embodiment 29

The health-care preparation according to Embodiment 28, characterized in that the dosage form for gastrointestinal administration is at least one of the following: powder, granule, pill, tablet, capsule, oral cream, paste, decoction, mixture, syrup, wine, and distillate.

### EXAMPLE 1

### Supercritical Extraction

Supercritical fluid (SF) refers to a fluid whose density is close to liquid, whereas its diffusion coefficient and viscosity are close to gas. In other words, the properties of a supercritical fluid are between gas and liquid, when some gas (liquid) or a mixture of gas (liquid) is under the condition that both the operation pressure and temperature are higher than their respective critical point. The supercritical fluid extraction (SFE) technology is a technology of extracting, by using a supercritical fluid as a solvent, and then separating some effective ingredients from the solid or liquid. The CO₂-supercritical fluid extraction is suitable for extraction of lipophilic substances with relatively small molecular weight. Herein, the CO₂-supercritical fluid extraction is suitable for extraction of fat or lipid components from the Nitraria fruit.

(1) A Nitraria fruit raw material was obtained.

The Nitraria fruit raw material can encompass fresh Nitraria fruit, dried Nitraria fruit, Nitraria fruit pulp, and/or Nitraria fruit seed. Lipid/fat components of the Nitraria fruit can be found in both the Nitraria fruit pulp and the Nitraria fruit seed, and the lipid/fat components contained in the two portions are slightly different. Therefore, the raw material for extraction can be a combination of one or two or more selected from the group consisting of the above four types (fresh fruit, dried fruit, fruit pulp, and/or fruit seed) as needed. In one example, before the extraction, the Nitraria fruit raw material was moderately smashed. In view of the capacity of the supercritical extraction equipment used in this example, about 10 g of the Nitraria fruit raw material was used for extraction.

(2) In the supercritical extraction equipment, the Nitraria fruit raw material was extracted for 2-3 h under a pressure of 20-35 MPa, at a temperature of 35-55° C., and at a CO₂ flow rate of 1-2 L/min.

The above extraction conditions were all optimized by experiments, as detailed below.

(2.1) Study of the extraction temperature and extraction pressure.

A single factor study was separately performed for the extraction temperature and the extraction pressure, respectively (see Table 1). The results show that an optimal extraction temperature in this experiment was 55° C. and an optimal pressure was 30 MPa. Under that condition, the extraction was performed for 2 h. The yield of the products reached 3.42%.

TABLE 1
Influence of the extraction temperature and extraction pressure on
the yield of fat-soluble products (CO₂ flow rate = 1 L/min).

| Temperature | | |
|---|---|---|
| 35° C. | 45° C. | 55° C. |

| P (MPa) | 25 | 30 | 35 | 25 | 30 | 35 | 25 | 30 | 35 |
|---|---|---|---|---|---|---|---|---|---|
| Yield | 2.19 | 2.49 | 2.31 | 2.36 | 2.72 | 3.35 | 2.41 | 3.42 | 3.27 |
| (%) | | | | | | | | | |

(2.2) Study of the CO₂ flow rate.

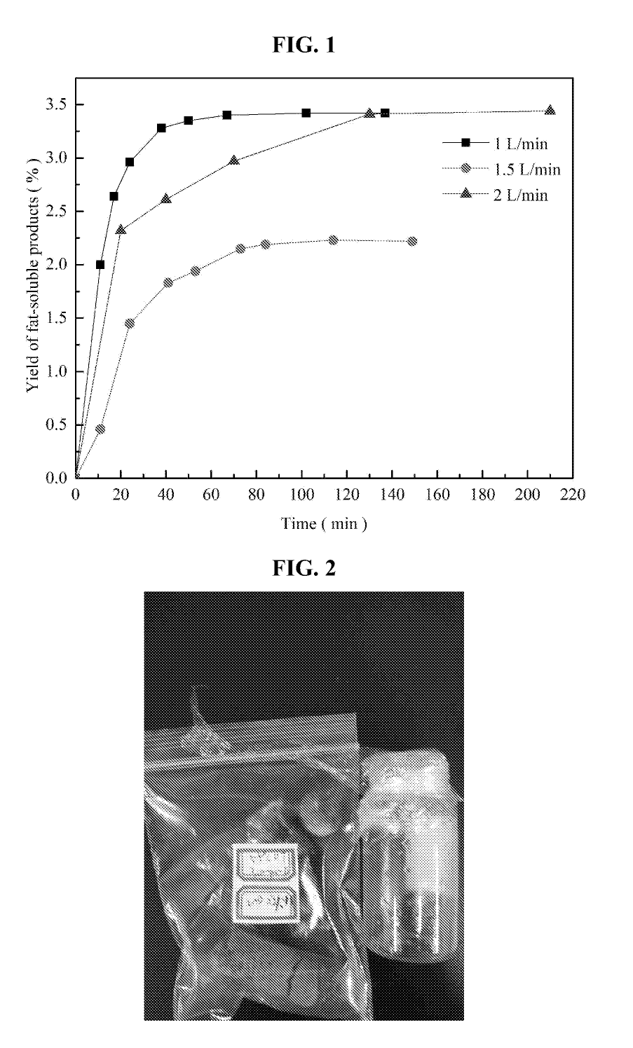

At an extraction temperature of 55° C. and under a pressure of 30 MPa, the influence of the CO₂ flow rate on the yield of oily extracts was studied. Four values of CO₂ flow rates were used, i.e., 0.5, 1, 1.5, and 2 L/min. The statistical analysis of experimental data in the 0.5 L/min group was discarded, because the extraction speed was too slow due to the low flow rate. FIG. 1 shows the change trend in yield with the extraction time at the other three flow rates. As shown in the figure, a higher extraction ratio (3.42%) can be reached at CO₂ flow rate of about 1 L/min within a relatively short period of time (>100 min).

(3) Oil-like extract was separated from the defatted sample (sometimes referred to as the defatted residues) obtained by the extraction.

FIG. 2 shows products obtained from step (2), and the oily extract obtained by the supercritical extraction had a faint yellow color, and the dark red solid in the bag on the left side is the powder obtained by supercritical extraction of the obtained defatted residues and then drying.

(4) The defatted residues were fully infiltrated with an entrainer, and then extracted, in the supercritical extraction equipment, for 1-3 h under a pressure of 20-35 MPa, at a temperature of 35-55, at a CO₂ flow rate of 1 L/min, and at an entrainer flow rate of 0.2-1.0 mL/min, to obtain an extracted solution.

In this example, the entrainer is a mixture of ethanol and water. The extraction conditions of this step were all optimized by experiments. The influences of the concentration of ethanol-water of the entrainer, the extraction temperature, the extraction pressure, the static soaking amount of the entrainer, and the dynamic flow rate of the entrainer were studied. Under all the following conditions, the extraction duration was for 3 h. Furthermore, products in the early stage of the extraction were discarded since they were obviously oil-containing. The early stage of the extraction mentioned here varies in time duration, depending upon the extraction conditions. To compare the active components of the extracts, anthocyanins and total polyphenols were selected as substances representative of active components, and the content of anthocyanins and the content of total polyphenols are measured. In this example, the content of anthocyanins in the extracted product was detected by pH-differential spectrophotometry, and the content of total polyphenols in the extracted product was detected by the Folin-phenol reagent method.

(4.1) Influence of the concentration of ethanol-water solution of the entrainer.

The entrainer and the defatted residues were statically soaked in a volume-to-mass ratio of (1 mL):(1 g), and then extracted at a temperature of 55° C. and under a pressure of 30 MPa, in order to study the influence of the concentration of ethanol-water solution on the yield of extracts. Meanwhile, the dynamic flow rate of the entrainer was controlled at 1 mL/min, and the CO₂ flow rate was controlled at 1 L/min. The results are as shown in Table 2. From the experimental results in Table 2, 65% ethanol was chosen an optimal concentration.

TABLE 2
Influence of the concentration of the entrainer (ethanol-water solution).

| | | Content of | Content of total |
|---|---|---|---|
| Concentration | | anthocyanins | polyphenols |
| of ethanol (v/v) | Yield (%) | (%) | (%) |
| 35% | 38.75 | 0.13 | 1.76 |
| 65% | 44.03 | 0.13 | 3.03 |
| 80% | 21.73 | 0.11 | 1.56 |
| 95% | 21.2 | 0.063 | 0.79 |

(4.2) Influence of the extraction temperature.

65% ethanol was used as the entrainer, and the entrainer and the defatted residues were statically soaked in a volume-to-mass ratio of (1 mL):(1 g), and then extracted under a pressure of 30 MPa, in order to study the influence of the extraction temperature on the yield of extracts. Meanwhile, the dynamic flow rate of the entrainer was controlled at 1 mL/min, and the CO₂ flow rate was controlled at 1 L/min. The results are as shown in Table 3.

From the results in Table 3, the temperature of 55° C. was chosen as an optimal temperature. Considering that a higher temperature would result in the increased energy consumption and the damaged active components, tests at higher temperatures were not performed.

TABLE 3
Influence of the extraction temperature.

| | | Content of | Content of total |
|---|---|---|---|
| T/° C. | Yield (%) | anthocyanins (%) | polyphenols (%) |
| 35 | 36.97 | 0.10 | 2.67 |
| 45 | 35.73 | 0.12 | 3.01 |
| 55 | 44.03 | 0.13 | 3.03 |

(4.3) Influence of the extraction pressure.

65% ethanol was used as the entrainer, and the entrainer and the defatted residues were statically soaked in a volume-to-mass ratio of (1 mL):(1 g), and then extracted at a temperature of 55° C., in order to study the influence of the extraction pressure on the yield of extracts. Meanwhile, the dynamic flow rate of the entrainer was controlled at 1 mL/min, and the CO₂ flow rate was controlled at 1 L/min. The results are as shown in Table 4.

Generally, the higher the extraction pressure, the more advantageous it is for the extraction of products. Table 4 shows that the highest yield can be reached under a pressure of 35 MPa. The content of anthocyanins and the content of total polyphenols did not vary much under different pressures. In view of the upper limit of the operating pressure of large-scale production equipment and the operating cost, tests under higher pressures were not performed.

TABLE 4
Influence of the extraction pressure.

| | | Content of | Content of total |
|---|---|---|---|
| P/MPa | Yield (%) | anthocyanins (%) | polyphenols (%) |
| 20 | 32.72 | 0.12 | 2.63 |
| 25 | 36.46 | 0.11 | 2.52 |
| 30 | 44.03 | 0.13 | 3.03 |
| 35 | 52.63 | 0.13 | 2.78 |

(4.4) Influence of the dynamic flow rate of the entrainer.

65% ethanol was used as the entrainer, and the entrainer and the defatted residues were statically soaked in a volume-to-mass ratio of (1 mL):(1 g), and then extracted at a temperature of 55° C. and under a pressure of 30 MPa, in order to study the influence of the dynamic flow rate of the entrainer on the yield of extracts. Meanwhile, the CO₂ flow rate was controlled at 1 L/min. The results are as shown in Table 5.

Theoretically, the higher the dynamic flow rate of the entrainer, the more advantageous it is for the extraction of products. This has been proved by the results as shown in Table 5. However, the increase in the flow rate will increase the usage amount of the entrainer, and thus the difficulty and cost of the subsequent processing. Therefore, the dynamic flow rate of the entrainer should be considered comprehensively.

In addition, there was a case in which the column was blocked during the experiment. Accordingly, an experiment in which the entrainer was statically soaked within the extraction column instead of using a dynamic entrainer was attempted. No product was brought out at the CO₂ flow rate in a large-scale test. Therefore, it was speculated that after the entrainer in the column was mixed with the material, the outlet of the column was blocked due to the excessive stickiness. In this process, a dynamic entrainer is indispensable.

TABLE 5
Influence of the dynamic flow rate of the entrainer.

| Flow rate of the | | Content of | Content of total |
|---|---|---|---|
| entrainer (mL/min) | Yield (%) | anthocyanins (%) | polyphenols (%) |
| 0.2 | 22.18 | 0.12 | 2.87 |
| 0.5 | 26.52 | 0.12 | 3.00 |
| 0.75 | 35.55 | 0.12 | 3.07 |
| 1 | 44.03 | 0.13 | 3.03 |

(4.5) Influence of the static soaking amount of the entrainer.

65% ethanol was used as the entrainer, and the entrainer and the defatted residues were extracted at a temperature of 55° C. and under a pressure of 30 MPa, in order to study the influence of the mixing ratio of the entrainer and the defatted residues (i.e., static soaking amount) on the yield of extracts. Meanwhile, the static soaking duration was controlled at 30 min, the dynamic flow rate of the entrainer was controlled at 1 mL/min, and the CO₂ flow rate was controlled at 1 L/min. The results are as shown in Table 6.

Table 6 indicates that the yield shows a rising trend as the static soaking amount increases. However, the influence is not very obvious. It is mainly because that the dynamic entrainer was also used at the same time. In view of the cost for large-scale production and the subsequent processing, a lower entrainer soaking amount can be selected.

In addition, considering that the entrainer also takes the sampling volume of the supercritical extraction equipment, 5 g of the defatted residues was selected for extraction.

TABLE 6
Influence of the static soaking amount of the entrainer.

| Static Soaking Amount of | | Content of | |
|---|---|---|---|
| the Entrainer (mL/g (raw | | anthocyanins | Content of total |
| material)) | Yield (%) | (%) | polyphenols (%) |
| 0 | 39.10 | 0.12 | 2.55 |
| 0.5 | 41.93 | 0.13 | 2.87 |
| 1 | 44.03 | 0.13 | 3.03 |

(4.6) Influence of the extraction duration.

65% ethanol was used as the entrainer, and the extraction was performed at a temperature of 55° C. and under a pressure of 30 MPa, in order to study the influence of the extraction duration on the yield of extracts. Meanwhile, the dynamic flow rate of the entrainer was controlled at 1 mL/min, and the CO₂ flow rate was controlled at 1 L/min. The results are as shown in Table 7.

During the experiment, the products obtained in the first 20 min were yellow, and discarded. The yield of those discarded products was not taken into the table. Table 7 shows that many products have been obtained when the extraction was performed for 40 min. However, the yield of products was significantly decreased after the extraction was performed for 185 min. In view of cost, it is appropriate to perform the extraction for 3 h.

TABLE 7
Influence of the extraction duration.

| | | | Content of | |
|---|---|---|---|---|
| Extraction | | | anthocyanins | Content of total |
| duration | Yield in | Total yield | in each stage | polyphenols in |
| (min) | stage (%) | (%) | (%) | each stage (%) |
| 40 | 14.21 | 14.21 | 0.12 | 3.17 |
| 60 | 9.84 | 24.05 | 0.13 | 2.83 |
| 94 | 8.00 | 32.05 | 0.15 | 2.87 |
| 185 | 10.71 | 42.76 | 0.09 | 0.061 |

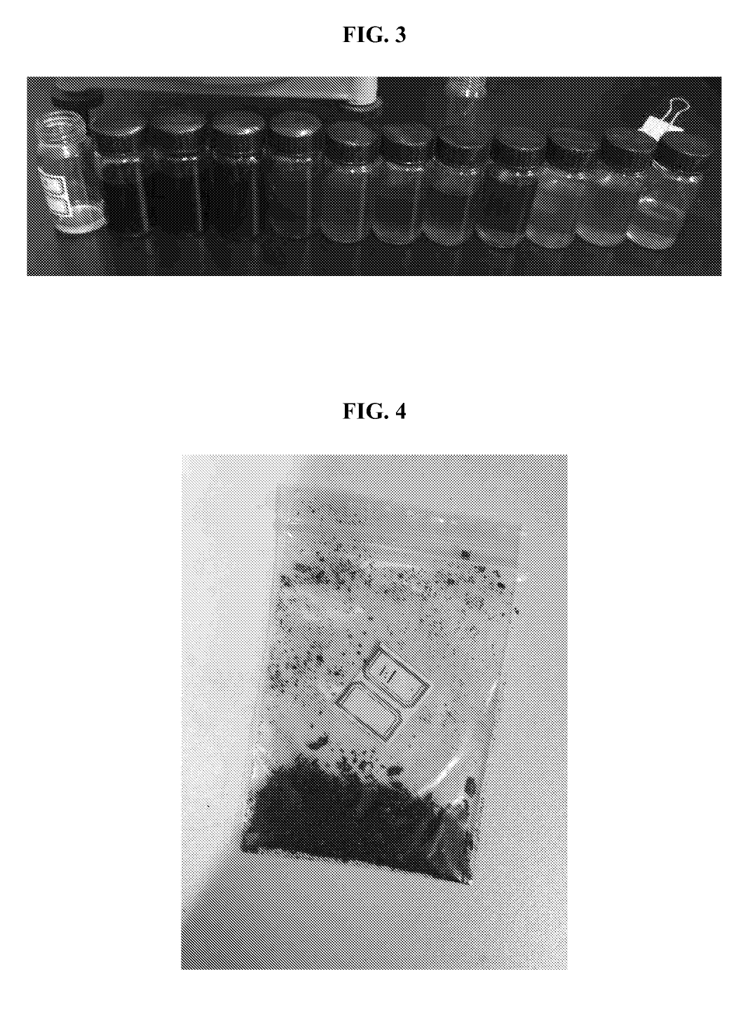

Taking those experimental results obtained by studies with a single factor, the yield of products is 44.03%, the content of anthocyanins is 0.13%, and the content of total polyphenols is 3.03%, when the extraction is performed for 3 h under the following conditions: concentration of ethanol of 65%; T=55° C.; P=30 MPa; static soaking amount of 1 mL/g (raw material); dynamic flow rate of the entrainer=1 mL/min; and CO₂ flow rate=1 L/min. FIG. 3 shows the collection bottles having the extracted solution therein, and the liquid in the second bottle from the left side is the extracted solution collected in the early extraction stage and is deep red. As the extraction duration increases, the liquid in the bottles becomes lighter in color. It is indicated by analysis that the extraction conditions mentioned above can extract anthocyanins and polyphenols from the Nitraria fruit raw material.

(5) The extracted solution was dried to obtain a powder extract.

The extracted solution can be stored conveniently and subjected to the subsequent processing only after it is dried. The drying process usually comprises the following steps:

(5.1) rotary evaporation: the extracted solution is rotatably evaporated under a reduced pressure to remove a solvent, at a temperature of 40-50° C. and under a pressure of −0.09 MPa as measured by a vacuum gauge; and

(5.2) freeze-drying: The product obtained by rotary evaporation is further freeze-dried to obtain the final product, at a freeze-drying temperature of −50° C. and under an absolute pressure of 5 Pa. The obtained product is deep red powder as shown in FIG. 4.

### EXAMPLE 2

### Solvent Extraction

The solvent extraction method has simple steps and can be performed for mass extraction. It is suitable for industrial large-scale production and application.

(1) Nitraria fruit raw material was obtained.

The Nitraria fruit raw material used in the solvent extraction method is the same as that used in the supercritical extraction method in the above example. The Nitraria fruit raw material can encompass fresh Nitraria fruit, dried Nitraria fruit, Nitraria fruit pulp, and/or Nitraria fruit seed. The raw material for extraction can be a combination of one or two or more selected from the group consisting of the above four types (fresh fruit, dried fruit, fruit pulp, and/or fruit seed) as needed. In one example, before the extraction, the Nitraria fruit raw material was moderately smashed.

(2) In the extraction equipment, the stirring extraction was performed with the Nitraria fruit raw material and 65% ethanol at a temperature of −55° C.

To achieve better extraction effect, at least one of the following extraction conditions can be optimized:

(2.1) the mass-to-volume ratio of the Nitraria fruit raw material to the 65% ethanol is adjusted, for example, to between about 1:3 and about 1:5;

(2.2) an appropriate stirring extraction duration is selected, for example, between about 1 h and about 2 h;

(2.3) the extraction is repeated, for example, for once or twice;

(2.4) pressure treatment is performed, and the pressurized pressure is can be between about 50 MPa and about 300 MPa; and

(2.5) ultrasonic vibration treatment is performed, and the power of the ultrasonic vibration treatment is about 600 W.

(3) A liquid extract was separated from solid residues obtained by the extraction.

(4) The liquid extract was purified and dried to obtain a powder extract.

The purpose of the purification process is to allow the extracted product to be convenient for the subsequent storage and processing, instead of removing other components present in the extracted product to make the extracted product have only a single component. The purifying process comprises at least one of the following:

(4.1) Rotary evaporation was performed at a temperature of 50° C. and under a reduced pressure, in order to remove the solvent in the liquid extract.

(4.2) Chromatography was performed by a macro-porous resin adsorption column to remove sugar in the liquid extract.

Further, anthocyanins and polyphenols can be adsorbed by AB-8 resin, and then anthocyanins and polyphenols adsorbed on the resin can be eluted with 65% ethanol, and the eluted solution can be directly sprayed into a powder after the ethanol is removed. After sugar is removed by the AB-8 macro-porous resin and thus the contents of anthocyanins and polyphenols are increased, the eluted solution has low viscosity and is non-hygroscopic and can be formed into powder, and the contents of anthocyanins and polyphenols in the form of powder are also increased, up to 2% (anthocyanins) and 40% (total polyphenols), respectively.

(4.3) Petroleum ether was used to perform extraction, to remove the resin present in the liquid extract.

(4.4) Ethanol was used to perform extraction, to remove the pectin present in the liquid extract.

(4.5) Acetone was used to perform extraction, to remove the sugar and polysaccharides present in the liquid extract.

(4.6) Protein present in the liquid extract was removed by salt fractionation or solvent precipitation.

Further, the drying process of the powder extract can be done by freeze-drying or spray-drying. The processing conditions for the two methods are well known to those skilled in the art and are not repeated here.

FIG. 5 provides exemplary steps of the solvent extraction method for preparing active Nitraria fruit extracts. Fresh Nitraria fruit or dried Nitraria fruit was soaked in 65% ethanol for 6 h, and circularly and ultrasonically extracted for 1 h, and the mass-to-volume ratio of the fruit pulp to 65% ethanol is 1:10. The pulp was separated from the seed after the pulp was softened, and the solid residues were removed by centrifugal filtering to obtain a liquid extract; the liquid extract was concentrated under a reduced pressure to remove the ethanol so as to obtain a concentrate; the concentrate was diluted with water, and finely filtered and then subjected to chromatography with AB-8 resin to remove part of sugar in the concentrate; the sugar-free extract was concentrated again under a reduced pressure, and finally spray-dried or freeze-dried, to obtain an active powder extract from the Nitraria fruit (similar to the state as shown in FIG. 4).

### EXAMPLE 3

### Comparison of Yield and Content of Active Components of Nitraria Fruit

The extraction of active extracts from the Nitraria fruit raw material based on the supercritical extraction method as described in Example 1 was done in accordance with conditions shown in Table 8 one by one, and the corresponding yield, content of anthocyanins and content of total polyphenols were obtained.

The results show that, under optimal extraction conditions, the yield of products, content of anthocyanins and content of total polyphenols are all higher when the defatted residues, which are highly defatted, are extracted again. In addition, prolonging the static soaking time and prolonging the dynamic extraction duration would be helpful to increase the yield and the content of total polyphenols. However, those two approaches have no obvious influence on the increase of the content of anthocyanins.

The extraction of active extracts from Nitraria fruit raw material based on the solvent extraction method as described in Example 2 was done in accordance with conditions shown in Table 9 one by one, and the respective yield, content of anthocyanins and content of total polyphenols were obtained.

The results show that, under optimal extraction conditions, the increase of the mass of the primarily extracted Nitraria fruit raw material, the increase of the ratio of the extraction agent (65% ethanol-water solution) and the increase of the extraction temperature are apparently helpful to increase the yield and the content of total polyphenols; crushing the Nitraria fruit raw material and the number of times of repeating the extraction have no obvious influence on the increase of the yield; and the ultrasonic vibration is helpful to increase the content of the obtained anthocyanins.

TABLE 8
Comparison of yield, content of anthocyanins and content of total polyphenols under various conditions for the supercritical extraction method.

| | Oil extraction conditions | | | | | | | | Dynamic | | | | | |
|---|---|---|---|---|---|---|---|---|---|---|---|---|---|---|
| | | | CO₂ | Extrac- | | | Entrainer | Static | flow rate | CO₂ | Extrac- | | content of | content of |
| | | Pres- | Flow | tion | | Pres- | (ethanol- | soaking | of the | Flow | tion | | antho- | total |
| | Temp. | sure | rate | duration | Temp. | sure | water | time | entrainer | rate | duration | yield | cyanin | polyphenols |
| No. | (° C.) | (MPa) | (L/min) | (h) | (° C.) | (MPa) | solution) | (min) | (L/min) | (L/min) | (h) | (%) | (%) | (%) |
| 1 | 35 | 20 | 0.5 | 1 | 55 | 20 | 65% | 15 | 1 | 1 | 3 | 32.7 | 0.12 | 2.63 |
| 2 | 55 | 35 | 2 | 3 | 55 | 30 | 95% | 30 | 1 | 1 | 3 | 21.2 | 0.063 | 0.79 |
| 3 | 55 | 30 | 1 | 3 | 55 | 35 | 65% | 30 | 1 | 1 | 3 | 52.6 | 0.13 | 2.78 |
| 4 | 55 | 30 | 1 | 2 | 55 | 30 | 65% | 30 | 1 | 1 | 3 | 41.9 | 0.13 | 2.87 |
| 5 | 55 | 30 | 1 | 2 | 55 | 30 | 65% | 0 | 1 | 1 | 3 | 39.1 | 0.13 | 2.55 |
| 6 | 55 | 30 | 1 | 2 | 55 | 30 | 65% | 30 | 0.0002 | 1 | 3 | 22.2 | 0.12 | 2.87 |
| 7 | 55 | 30 | 1 | 2 | 55 | 30 | 65% | 30 | 1 | 1 | 3 | 44 | 0.13 | 3.03 |
| 8 | 55 | 30 | 1 | 3 | 35 | 30 | 65% | 30 | 1 | 1 | 3 | 37 | 0.1 | 2.67 |
| 9 | 55 | 30 | 1 | 3 | 35 | 30 | 65% | 30 | 1 | 1 | 1 | 24.1 | 0.12 | 3.03 |

TABLE 9
Comparison of yield, content of anthocyanins and content of total polyphenols under conditions for the solvent extraction method.

| | | Extraction | Mixing | | Extraction | Times of | | Ultra | | | total |
|---|---|---|---|---|---|---|---|---|---|---|---|
| | Raw Nitraria | agent (ethanol- | ratio | T | duration | repeating | pressure | sonic | yield | anthocyanin | polyphenol |
| No. | material | water solution) | (m:v) | (° C.) | (h) | extraction | (MPa) | (W) | (%) | (%) | (%) |
| 1 | 10 g of fruit power | 65% | 1:5 | 55 | 1 | 0 | 0 | 0 | 61.3 | 0.12 | 5.68 |
| 2 | 10 g of fruit power | 65% | 1:5 | 35 | 1 | 0 | 0 | 0 | 55.4 | 0.12 | 3.72 |
| 3 | 10 g of fruit power | 65% | 1:5 | 55 | 1 | 1 | 0 | 0 | 62.5 | 0.12 | 5.62 |
| 4 | 10 g of full fruit | 65% | 1:3 | Room T | 1 | 0 | 0 | 0 | 22.6 | 0.11 | 2.12 |
| 5 | 10 g of full fruit | 65% | 1:3 | Room T | 1 | 1 | 0 | 0 | 35.1 | 0.11 | 2.2 |
| 6 | 10 g of full fruit | 65% | 1:3 | 35 | 1 | 0 | 0 | 0 | 28.7 | 0.12 | 2.32 |
| 7 | 10 g of full fruit | 65% | 1:3 | 35 | 1 | 1 | 0 | 0 | 41.8 | 0.13 | 2.44 |
| 8 | 100 g of fruit power | 65% | 1:2 | 55 | 10 min | 0 | 300 | 0 | / | 0.14 | 5.77 |
| 9 | 500 g of fruit power | 65% | 1:10 | 45 | 1 | 0 | 0 | 600 | / | 2.107 | 36.175 |

The two extraction methods are compared. By the supercritical extraction method, the oil-like extract, which cannot be obtained by the solvent extraction method, can be obtained. By the solvent extraction method, the resulting yield and the content of total polyphenols are significantly higher than those obtained by the supercritical extraction method. However, the two extraction methods have no obvious difference in the extraction of anthocyanins. In view of production energy consumption, obtaining a larger amount of anthocyanins by the solvent extraction method is more suitable to meet the requirement of practical production.

### EXAMPLE 4

### Pharmaceutical Compositions and/or Health-care Preparations Containing Active Nitraria Fruit Extracts

As shown in FIG. 6, extracts containing different active components can be obtained from the Nitraria fruit raw material by the two extraction methods. For example, oil-like extracts, defatted residues, and other polar components (such as flavonoids and/or alkaloids) can be obtained by the supercritical extraction method, and polar extracts dissolved in ethanol and solid residues can be obtained by the solvent extraction method. Those extracts are all biologically active. Especially for the extracts obtained by the two non-toxic and moderate extraction methods, i.e., the supercritical extraction method and the solvent extraction method, the biological activities of the Nitraria fruit can be maximally retained. When one or two or more of those extracts are combined with each other or with other pharmaceutical ingredients, pharmaceutical compositions used for treating or preventing certain diseases or symptoms can be formed.

For example, a pharmaceutical composition used for preventing and/or treating lower urinary tract symptoms due to benign prostatic hypertrophy, for example, the pharmaceutical composition each, further comprises at least one of the following: about 0.01-100 g of lycopene extract, about 0.01-10 g of saw palmetto extract, about 0.001-100 g of pumpkin seed extract, and about 0.01-0.5 g of Selenium (Se).

In one example, the pharmaceutical composition each further comprises at least one of the following ingredients: about 0.01-10 g of Lyceum ruthenicum or its extract, about 0.01-10 g of black tomato or its extract, about 0.01-100 g of pumpkin seed, about 0.01-10 g of protein extract of pumpkin seed, about 0.01-100 g of lentinan, about 0.01-100 g of Pleurotus ostreatus polysaccharide, about 0.01-100 g of agaric polysaccharide, about 0.01-100 g of Flammulina velutipes polysaccharide, about 0.01-100 g of Nitraria fruit polysaccharide, and about 0.01-100 g of spirulina extract.

Another example is a pharmaceutical composition used for preventing and/or treating an eye conditional or disease such as a macular degeration, especially macular degeneration associated with radiation of radioactive rays or radiation of ultraviolet rays and age-related macular degeneration. In one example, the pharmaceutical composition each further contains about 0.01-10 g of lutein, about 0.001-10 g of zeaxanthin, and about 0.001-10 g of astaxanthin.

The pharmaceutical composition can be used for regulating and restoring the immune function. In one example, the pharmaceutical composition can be combined with pharmaceutical ingredients for treating cancers.

The active Nitraria fruit extracts can also be used as health-care preparations for daily use by the healthy population or sub-healthy population. The health-care preparations contain one or two or more of those active Nitraria fruit extracts which are compatible with others. The optional implementations of the health-care preparations comprise the following: a health-care preparation containing one or two or more of those active Nitraria fruit extracts, and fresh Nitraria fruit juice; a liquid beverage containing one or two or more of those active Nitraria fruit extracts, and the total content of the active Nitraria fruit extracts in each liquid beverage is between about 0.1g and about 100 g; a liquid beverage containing one or two or more of those active Nitraria fruit extracts, and the total content of the active Nitraria fruit extracts in each liquid beverage is between about 0.1 g and about 1000 g; a health-care preparation containing an oil-like extract, and the oil-like extract is extracted from Nitraria fruit seed; a health-care preparation containing an oil-like extract, and the oil-like extract is extracted from Nitraria fruit pulp; and a health-care preparation containing an oil-like extract, and the oil-like extract is extracted from intact full Nitraria fruits.

Further, the health-care preparation can comprise excipients for forming a dosage form for gastrointestinal administration. The dosage form for gastrointestinal administration includes, but is not limited to, powder, granule, pill, tablet, capsule, oral cream, paste, decoction, mixture, syrup, wine, and distillate. It should be understood by those skilled in the art that the ratio of the contained excipients and active components is different for a different dosage form. Therefore, as a result, the content of the active Nitraria fruit extracts and the ratio of the components are different in different dosage forms. Rational selection is required according to the specific indications, during the production. Table 10 below shows the reference content of the active Nitraria fruit extracts in some dosage forms.

TABLE 10
Nitraria fruit extract dosage forms.

| | Unit content of the active |
|---|---|
| Dosage form | Nitraria fruit extracts (g) |
| tablet | 0.01-100 |
| powder | 0.01-1000 |
| cream | 0.01-1000 |
| paste | 0.01-1000 |
| hard capsule | 0.01-100 |
| soft capsule | 0.01-100 |
| granule pill and drop pill | 0.001-10 |
| large pill | 0.01-1000 |

In conclusion, the method for preparing active Nitraria fruit extracts in the present disclosure has mild conditions, high extraction efficiency, and well retained activities of the extracts, and the obtained extracts have broad medicinal and health-care promotion prospect.

### EXAMPLE 5

### Therapeutic Use of Nitraria tangutorum Bobr. Extract for Lower Urinary Tract Symptoms (LUTS) Related to Benign Prostatic Hyperplasia (BPH)

Lower urinary tract symptoms (LUTS) related to benign prostatic hyperplasia (BPH) are the consequence not only of a static enlargement but also of dynamic processes causing an exaggerated prostate smooth muscle contraction (Roehrborn & Schwinn, Alphal-adrenergic receptors and their inhibitors in lower urinary tract symptoms and benign prostatic hyperplasia, J Urol 2004, 171:1029-1035). In fact, an important proportion of patients diagnosed for overactive bladder have BPH as underlying condition but not necessarily linked to bladder outlet obstruction (Blaivas et al., Differential diagnosis of overactive bladder in men, J Urol 2009, 182:2814-2817). In this sense, decreasing smooth muscle tone in the bladder and prostate is an important strategy for medical treatment of LUTS related to BPH (Hennenberg et al., Pharmacology of the lower urinary tract, Indian J Urol 2014, 30:181-188).

Nitraria tangutorum fruits contain significant amounts of anthocyanins (Zheng et al., Anthocyanins composition and antioxidant activity of two major wild Nitraria tangutorum Bobr. variations from Qinghai-Tibet plateau, Food Res Int 2011, 44:2041-2046; Ma et al., In vitro and in vivo biological activities of anthocyanins from Nitraria tangutorum Bobr. fruits, Food Chem 2016, 194: 296-303; Rana et al., Total antioxidant capacity and characterization of Nitraria tangutorum fruit extract by rapid bioassay-directed fractionation, J AOAC Int 2016, 99:1219-1222). Anthocyanins have been demonstrated to induce vasodilation and the relaxation of other smooth muscle preparations, an effect likely mediated through the NO/cGMP pathway (Fumagalli et al., From field to health: a simple way to increase the nutraceutical content of grape as shown by NO-dependent vascular relaxation, J Agric Food Chem 2006, 54:5344-5349; Bell & Gochenaur, Direct vasoactive and vasoprotective properties of anthocyanin-rich extracts, J Appl Physiol 2006, 100(4):1164-70; Matsumoto et al., Delphidine-3-rutinoside relaxes the bovine ciliary smooth muscle through activation of ETB receptor and NO/cGMP pathway, Exp Eye Res 2005, 80:313-322). In addition, evidences supporting oral bioavailability of these compounds have been reported (Fang, Bioavailability of anthocyanins, Drug Metab Rev 2014, 46: 508-520; Bhaswant et al., Cyanidin 3-glucoside improves diet-induced metabolic syndrome in rats, Pharmacol Res 2015, 102:208-217). Pharmacological agents enhancing NO/cGMP such as phosphodiesterase type 5 (PDE5) inhibitors have shown clinical efficacy in the treatment of BPH-induced urinary symptoms (Yokoyama et al., Tadalafil for urinary tract symptoms secondary to benign prostatic hyperplasia: a review of clinical data in Asian men and an update on the mechanism of action, Ther Adv Urol 2015, 7:249-264), suggesting that NO/cGMP pathway activation could modulate smooth muscle tone of prostate and bladder neck (Angulo et al., Tadalafil enhances the inhibitory effects of tamsulosin on neurogenic contractions of human prostate and bladder neck, J Sex Med 2012, 9:2293-2306) and would have therapeutic relevance in the treatment of symptoms of BPH.

Based on these fundamentals the evidences supporting a potential role of N. tangutorum Bobr extract on the management of BPH symptoms are exposed below.

1) N. tangutorum Bobr. Extract (NtB) Inhibits Proliferation of Human Prostate Cancer Cells.

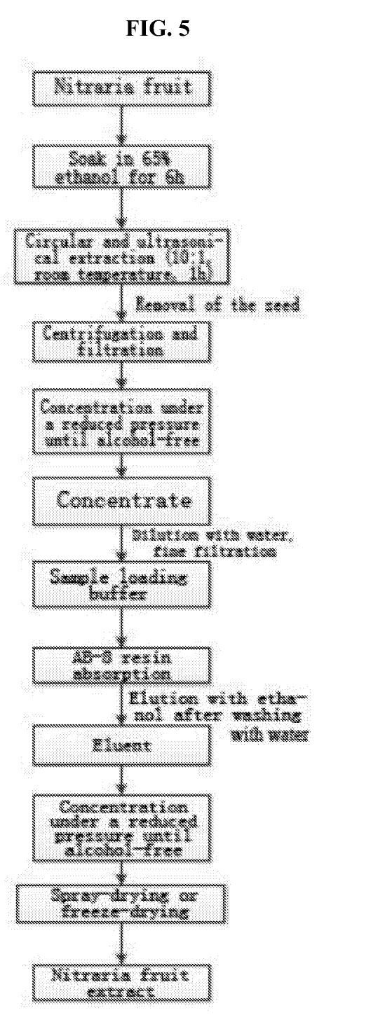

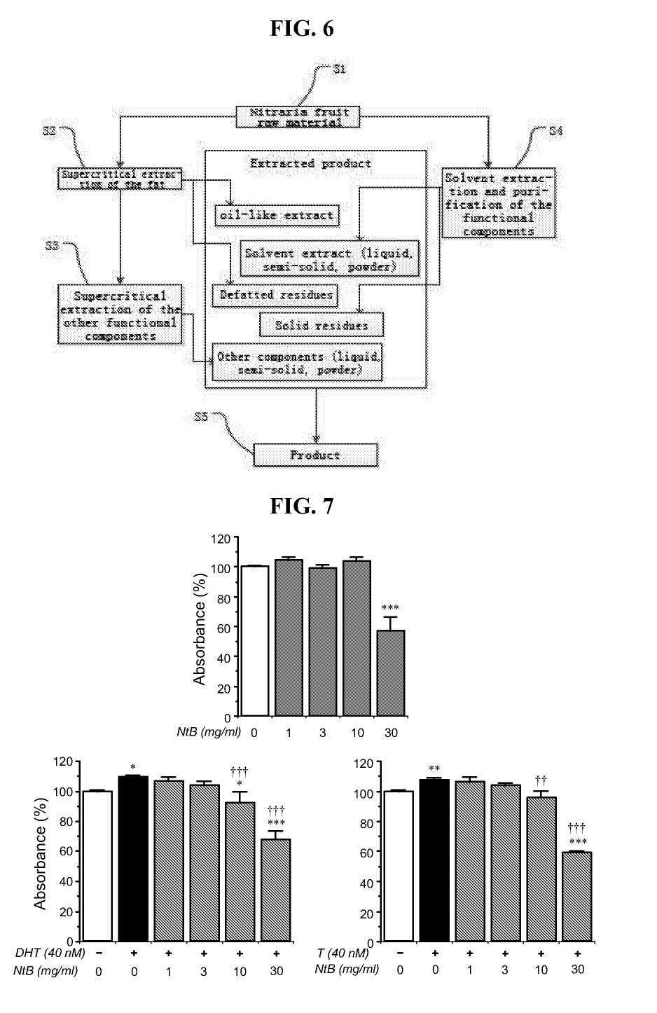

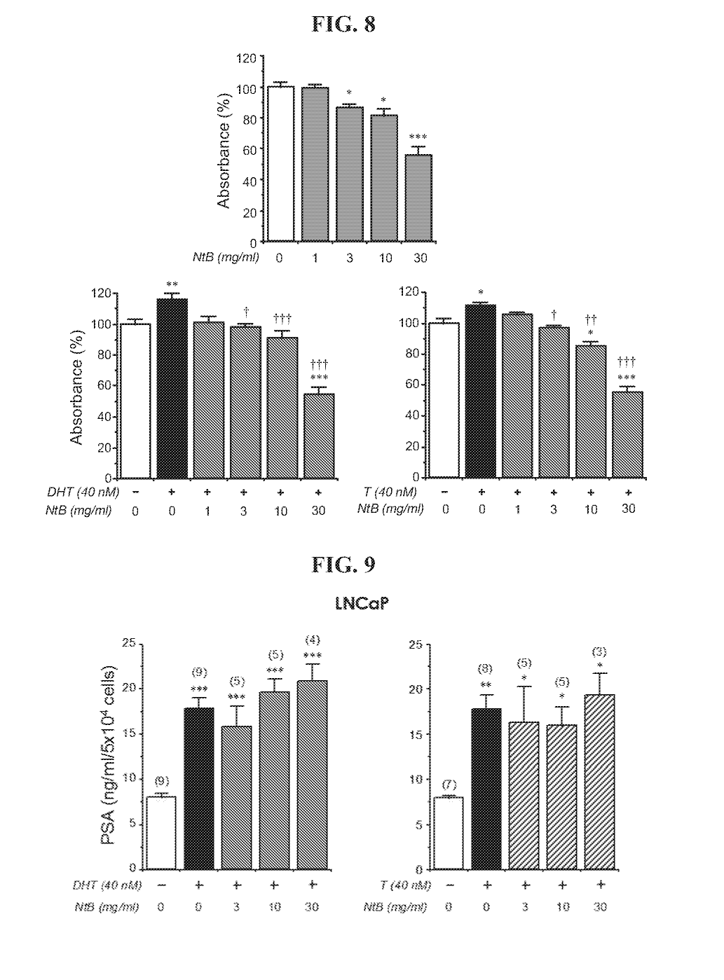

As shown in FIG. 7 and FIG. 8, NtB concentration-dependently inhibits proliferation of human prostate cancer cells. In fact it is able to cause such an effect in both androgen-sensitive (LNCaP) and androgen-insensitive (PC-3) human prostate cell lines. This evidence, together with the fact that NtB inhibits proliferation in the absence of androgen stimulation (either with testosterone or dihydrotestosterone) suggests that NtB exerts anti-proliferative effects independently of androgen signaling. This is further supported by the lack of interference of NtB on androgen-stimulated prostatic specific antigen (PSA) production in LNCaP cells (FIG. 9).

2) NtB Effectively Relaxes Human Prostate and Bladder Neck.

FIG. 10 shows the consistent, concentration-dependent, relaxant effect driven by addition of NtB on norepinephrine-contracted human prostate or bladder neck strips obtained from patients with BPH/LUTS.

3) NtB Stimulates NO/cGMP Pathway in Human Prostate and Bladder Neck.

Relaxant capacity of NtB is related to its ability to cause a significant accumulation of cGMP in both human prostate and bladder neck tissues (FIG. 11) suggesting that relaxation driven by NtB is mediated by NO/cGMP pathway stimulation. In fact, this suggestion is consistent with the capacity of NtB (10 mg/ml) to markedly enhance relaxation of human prostate and bladder neck strips induced by exposure to the PDE5 inhibitor tadalafil (FIG. 12). This evidence, not only reinforces the mechanistic properties of NtB as a NO/cGMP stimulator in these tissues but also suggest a therapeutic potential of NtB as an enhancer of the pharmacological activity of a drug, tadalafil, approved for BPH/LUTS and ED in Europe, USA and South Korea.

4) Acute Intraduodenal and Chronic (e.g., for 2 weeks) Oral Administration of NtB Improves Urodynamic Parameters in BPH In Vivo Model.

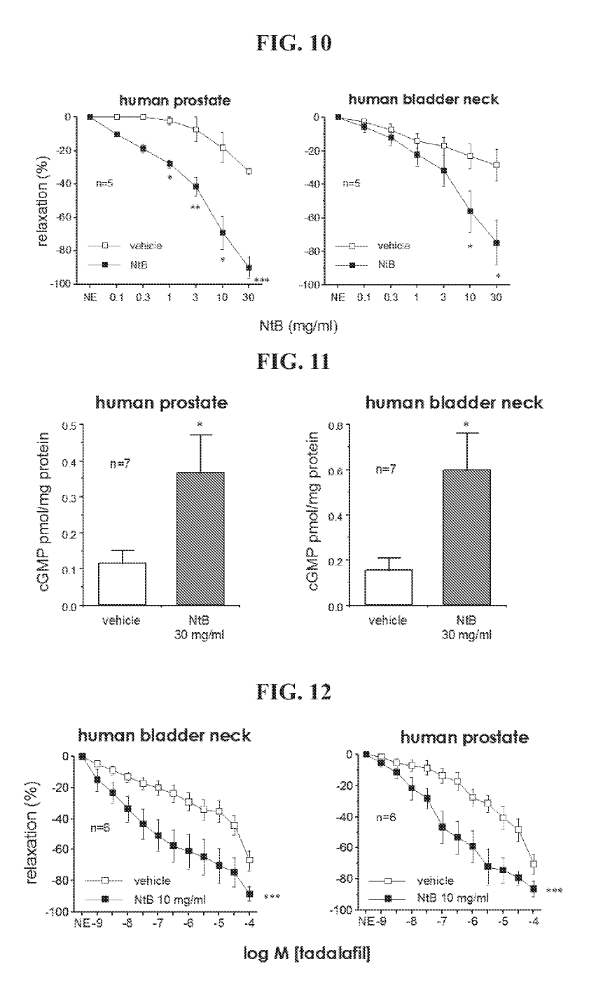

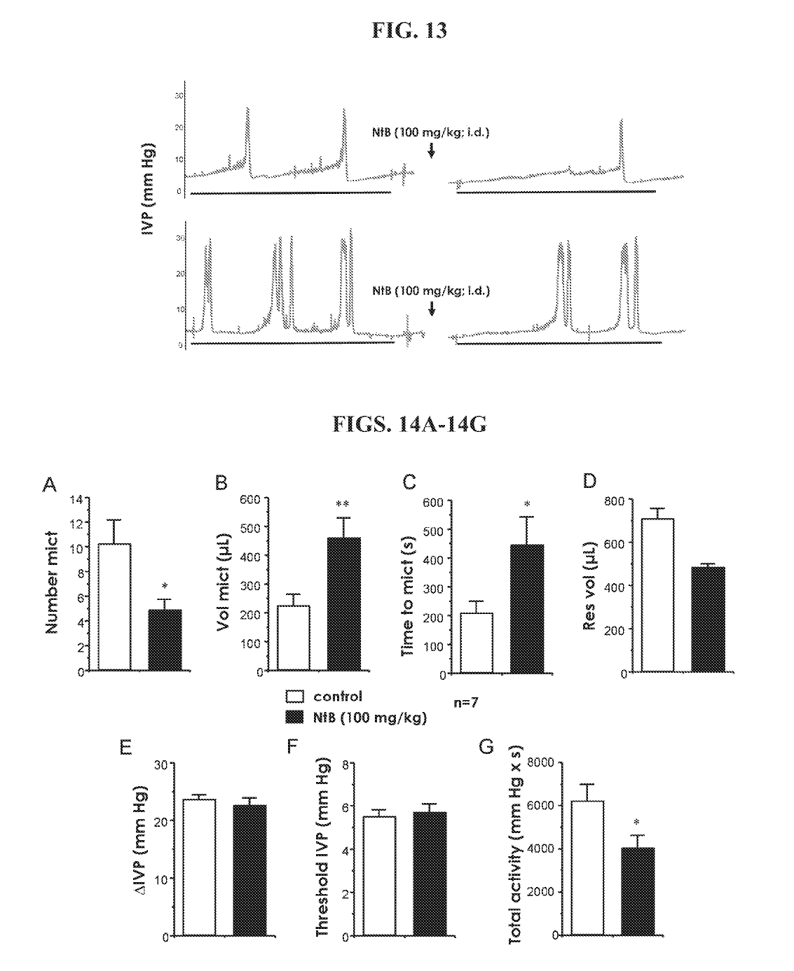

For in vivo relevance confirmation of in vitro results, the effects of NtB were evaluated in a rat model of BPH. This model uses castration of young rats (5-6 weeks old) followed by testosterone supplementation (5 mg/kg; s.c.) that leads to prostate hypertrophy. Urodynamic alterations in these animals can be observed after 3 weeks of testosterone supplementation (Liu et al., Amlodipine alone or combined with terazosin improves lower urinary tract disorder in rat models of benign prostatic hyperplasia, BJU Int 2009, 104:1752-1757). Urodynamic parameters were determined in rats after 5 weeks of testosterone supplementation before and 20 min after intraduodenal administration of NtB (100 mg/kg) by performing cystometries with infusion of 0.9% NaCl solution for 20 min into the bladder. Tracings of intravesical pressure (IVP) recordings showed that acute NtB administration reduced the activity of the bladder in BPH-induced rats (FIG. 13). Quantification of urodynamic parameters showed that NtB administration produced reduction of number of micturitions for the 20 min infusion period (FIG. 14A) while accordingly increased the micturition volume (FIG. 14B) and the time of infusion required for displaying the first micturition reflex (FIG. 14C). NtB administration also reduced the bladder containing residual volume after last micturition (FIG. 14D). Acute NtB did not modify IVP increase during micturition reflex (FIG. 14E) and did not influence the IVP threshold for developing a micturition reflex (FIG. 14F). As a global data, acute NtB administration significantly reduced the total activity of the bladder during the 20 min infusion period (measured as the area under the curve of the IVP) (FIG. 14G). Intraduodenal administration of NtB was not associated with modification of mean arterial pressure (MAP, in mm Hg) showing MAP values of 102.54±4.05 vs. 96.27±4.37 mmHg, n=7) or heart rate (HR, in bpm) showing HR values of 344±12 vs. 323±9 bpm, n=7). Thus, FIGS. 14A-14G show an improvement on urodynamic parameters in testosterone-supplemented rats after acute intraduodenal administration of NtB. This in vivo evidence is consistent with the in vitro demonstrated activities of NtB and supports the potential therapeutic relevance of NtB for the treatment of BPH.

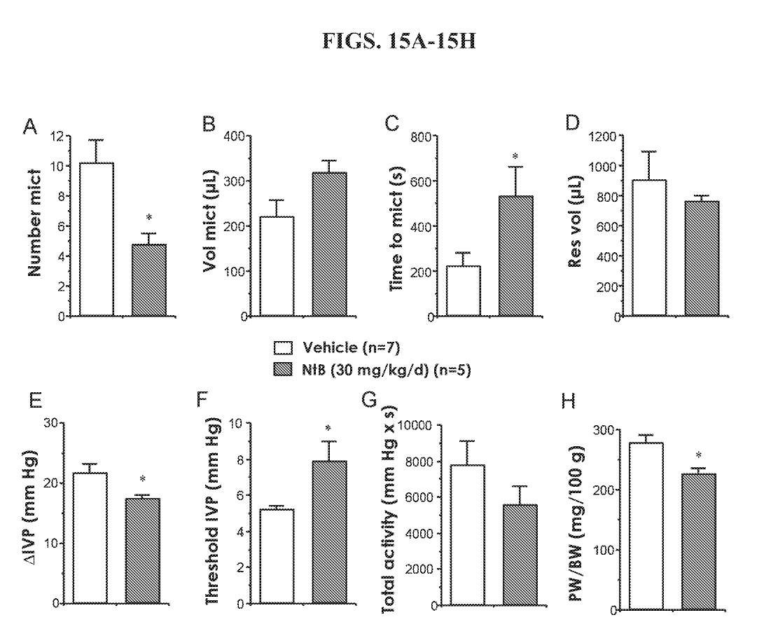

Using the same in vivo BPH model, it was also demonstrated that continuous oral administration of NtB for 2 weeks (30 mg/kg/d) to castrated and testosterone-injected rats improved urodynamic parameters in these animals. FIGS. 15A-15G show effects of oral administration of NtB for 2 weeks on urodynamic parameters in testosterone-supplemented rats. The two-week oral treatment with NtB reduced number of micturitions (FIG. 15A), increased micturition volume (FIG. 15B), increased the time of infusion required for displaying the first micturition reflex (FIG. 15C) and reduced the residual urine volume in the bladder (FIG. 15 D) but also reduced the increase in IVP during micturition reflex (FIG. 15E) and increased the IVP threshold for developing a micturition reflex (FIG. 15F). The total activity of the bladder was also reduced (FIG. 15G). The two-week oral administration of NtB was not associated with significant modification of mean arterial pressure (showing MAP values of 92.11±5.26 vs. 80.71±10.18 mmHg, n=7 for vehicle and n=5 for NtB, respectively) or heart rate (showing heart rate values of 318±12 vs. 316±40 bpm, n=7 for vehicle and n=5 for NtB, respectively). In addition to its effects on urodynamic function, the two-week oral treatment with NtB was associated with a reduction in prostate hypertrophy, as indicated by the significant reduction in the prostate weight/body weight ratio (FIG. 15H). Favourable urodynamic and prostatic effects by chronic oral NtB extract further support the therapeutic potential of this extract in the treatment of BPH.

### EXAMPLE 6

### Combination of Nitraria tangutorum Bobr. Extract and Lycopene for Use in BPH/LUTS

In this example, lycopene is used in addition to N. tangutorum extract for BPH/LUTS.

Results in Example 5 suggest that N. tangutorum Bobr extract does not interfere with androgen stimulation in the prostate. Thus, addition of an additional compound with anti-androgen activity would reasonably increase the potential activity of N. tangutorum Bobr extract to relieve benign prostatic hyperplasia (BPH)-induced symptoms. In this sense, lycopene has been shown to inhibit prostate cell proliferation and PSA production by reducing androgen-stimulated signaling (Herzog et al., Lycopene reduced gene expression of steroid targets and inflammatory markers in normal rat prostate, FASEB J2005, 19:272-274; Liu et al., Lycopene inhibits IGF-I signal transduction and growth in normal prostate epithelial cells by decreasing DHT-modulated IGF-I production in co-cultured reactive stromal cells, Carcinogenesis 2008, 29:816-823; Zhang et al., Effect of lycopene on androgen receptor and prostate-specific antigen velocity, Chin Med J (Engl) 2010, 123:2231-2236). Combination of N. tangutorum Bobr extract and lycopene could represent a strategy for treating both dynamic and structural alterations responsible for BPH-induced symptoms. This hypothesis is supported by the following results.

1) Lycopene Inhibits Androgen-Induced Proliferation of Androgen-Sensitive Human Prostate Cancer Cells.

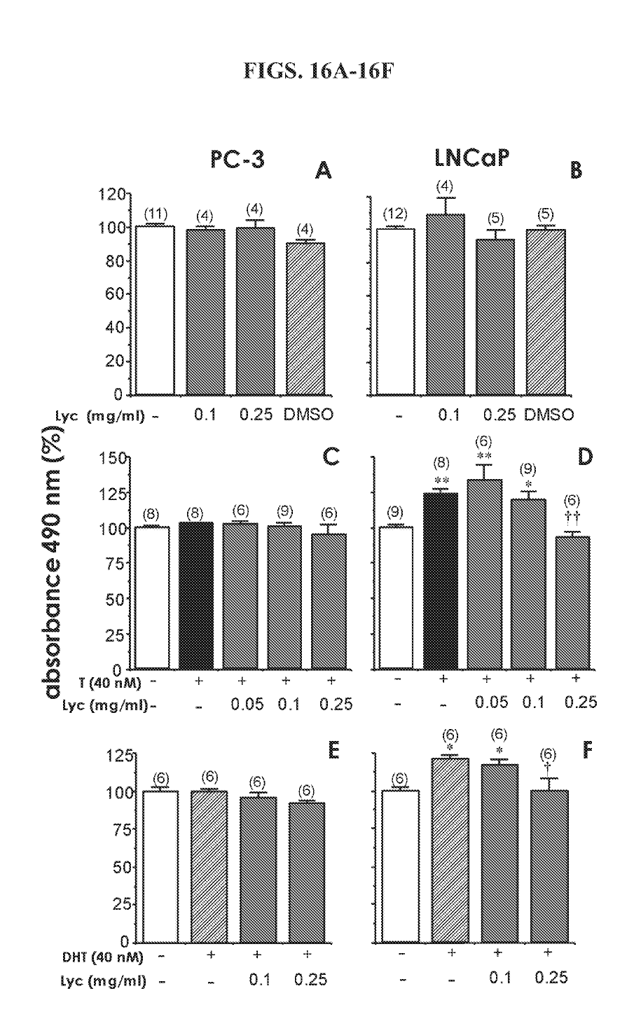

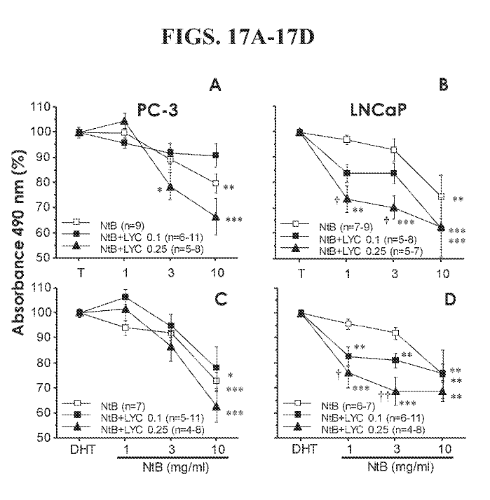

Exposure to lycopene (0.1 and 0.25 mg/ml) did not significantly influence proliferation of either PC-3 or LNCaP human prostate cancer cells in the absence of androgen stimulation (FIG. 16A and FIG. 16B). However, at a 0.25 mg/ml final concentration, lycopene effectively reversed testosterone (T)- or dihydrotestosterone (DHT)-induced increase in proliferation in androgen-sensitive LNCaP cells (FIG. 16D and FIG. 16F). In contrast, even in the presence of T or DHT lycopene failed to significantly reduce proliferation of androgen-insensitive PC-3 cells (FIG. 16C and FIG. 16E).

2) Lycopene Enhances Anti-Proliferative Activity of N. tangutorum Extract in Androgen-Sensitive Human Prostate Cancer Cells.

Anti-proliferative activity of NtB extract (1, 3 and 10 mg/ml) was not significantly influenced by lycopene co-administration (0.1 and 0.25 mg/ml) in PC-3 cells treated with DHT (FIG. 17C) and was only enhanced at 3 mg/ml NtB by 0.25 mg/ml lycopnene addition in T-treated PC-3 cells (FIG. 17A). In contrast, the potentiation of anti-proliferative activity of NtB by lycopene was clearly manifested in androgen-sensitive LNCaP cells exposed to T or DHT. Lycopene (0.1 and 0.25 mg/ml) produced a concentration-dependent potentiation of NtB-induced anti-proliferative effects in LNCaP treated with T which was clearly significant with lycopene at 0.25 mg/ml concentration and NtB at both 1 and 3 mg/ml concentrations (FIG. 17B). Similar effects were observed in DHT-treated LNCaP but, in this case, the effects were significant also with lycopene 0.1 mg/ml concentration and significantly more marked at 0.25 mg/ml concentration of lycopene (FIG. 17D). Thus, co-administration of lycopene increased sensitivity of androgen-sensitive LNCaP to the antiproliferative activity displayed by NtB extract. Considering that prostate hypertrophy in BPH is sensitive to anti-androgenic modulation, lycopene would enhance the potential inhibitory effects of NtB on prostate growth in BPH.

3) Lycopene Reduces Androgen-Stimulated PSA Production in Androgen Sensitive Human Prostate Cancer Cells (LNCaP).

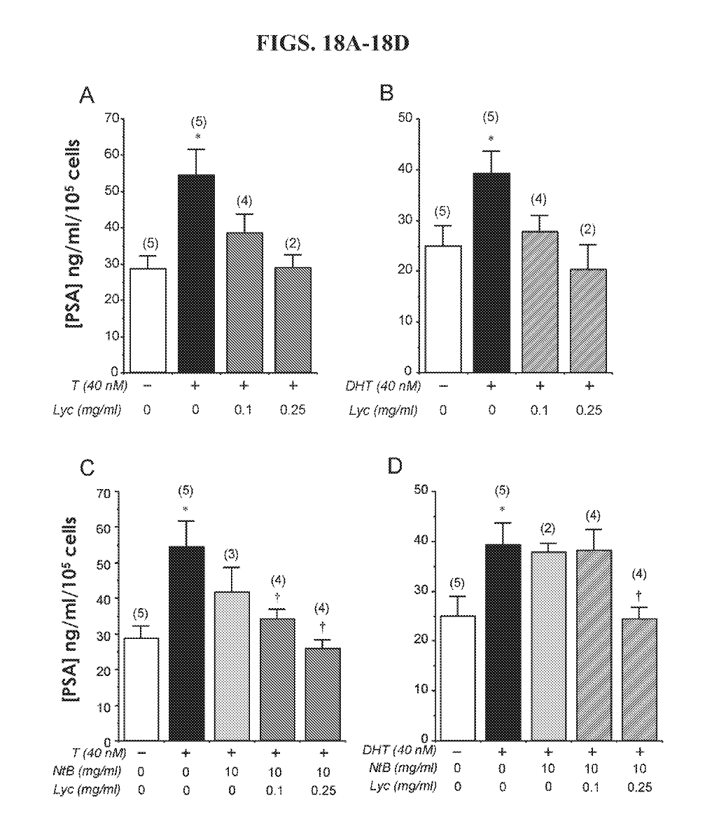

The increase in PSA production induced by androgens, either testosterone or dihydrotestosterone (40 nM), was prevented by treating of LNCaP cells with lycopene (FIGS. 18A-18B). The effects driven by lycopene on PSA production seems to be concentration-related as the higher concentration (0.25 mg/ml) displays greater inhibitory effects on PSA levels. In addition, the effects of lycopene on androgen-stimulated PSA production in LNCaP are still manifested when the cells are co-treated with the NtB extract. FIGS. 18C-18D shows that NtB alone does not significantly reduced testosterone- or dihydrotestosterone-induced PSA production but the combined addition of lycopene, mainly at the higher concentration (0.25 mg/ml), effectively reduces PSA production. This evidence propose additional activities of the combination of lycopene with NtB extract over NtB extract alone with respect to the ability to inhibit androgen-induced effects on prostate cells.

4) Lycopene Does Not Preclude NtB-Induced Relaxation in Human Bladder and Prostate and Produced by Itself Relaxation of These Tissues.

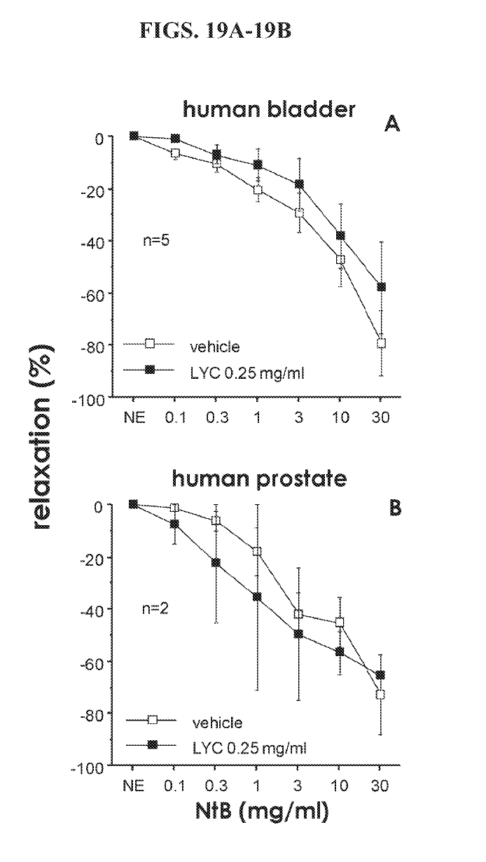

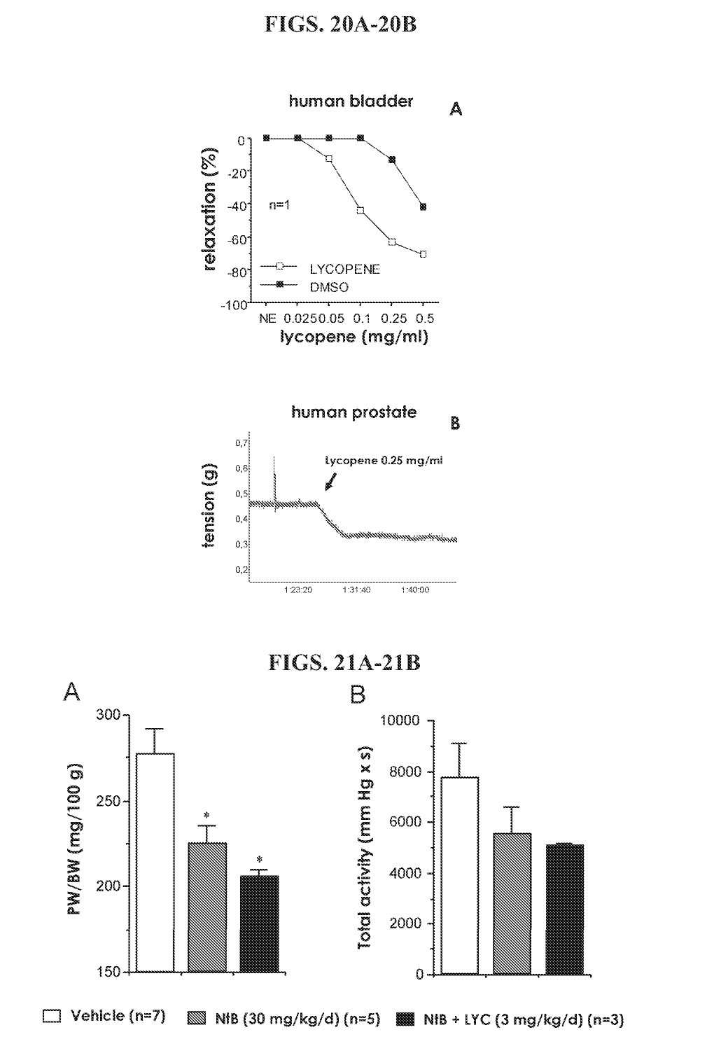

The presence of lycopene at an effective concentration in proliferation assays (0.25 mg/ml) failed to significantly alter the relaxant activity of NtB on human bladder and prostate tissues (FIG. 19A and FIG. 19B). This means that the positive effects of combining lycopene with NtB for reducing prostate proliferation are not hampered by a possible interference on the relaxant activity of NtB extract in human bladder and prostate. Furthermore, experiments suggest that lycopene could have relaxant activity by itself on these tissues (FIG. 20A and FIG. 20B).

5) Addition of Oral Lycopene to Oral NtB Administration Further Reduced Prostate Hypertrophy and Bladder Activity in Testosterone-Exposed Castrated Rats.

A two-week daily oral treatment with NtB extract (30 mg/kg/d) reduced the prostate weight/body weight ratio which is an index of prostate hypertrophy in the BPH rat model but this ratio was further reduced when combined oral treatment with lycopene was added. In addition, the combination of NtB with daily lycopene (3 mg/kg/d) tended to produce a greater reduction of the bladder activity. FIG. 21A and FIG. 21B show effects of oral administration of NtB plus lycopene for 2 weeks on prostate hypertrophy and bladder activity in testosterone-supplemented rats. These in vivo results (n=3) reinforce the idea that adding lycopene to the treatment with NtB extract would increase its therapeutic potential in the treatment of BPH.

### EXAMPLE 7

### Use of N. tangutorum Bobr extract in ED (Erectile Dysfunction)

Although the underlying processes are not fully understood, it has been proposed that BPH/LUTS and ED share pathophysiological mechanisms (Gacci et al., Critical analysis of the relationship between sexual dysfunctions and lower urinary tract symptoms due to benign prostatic hyperplasia, Eur Urol 2011, 60:809-825). This is supported by the consistent epidemiologic evidences establishing an independent link between LUTS/BPH and ED (Kirby et al., Erectile dysfunction and lower urinary tract symptoms: a consensus on the importance of co-diagnosis, Int J Clin Pract 2013, 67:606-618). In fact, one of the proposed alterations shared by both pathological conditions is an impairment of the NO/cGMP pathway (Park et al., Urinary tract symptoms (LUTS) secondary to benign prostatic hyperplasia (BPH) and LUTS/BPH with erectile dysfunction in Asian men: A systematic review focusing on tadalafil, World J Mens Health 2013, 31:193-207; Gacci et al., Critical analysis of the relationship between sexual dysfunctions and lower urinary tract symptoms due to benign prostatic hyperplasia, Eur Urol 2011, 60:809-825).

1) NtB Stimulates NO/cGMP Pathway.

Considering that drugs amplifying NO/cGMP signaling such as PDE5 inhibitors act at both prostate/bladder as well as penile smooth muscle levels, it could be speculated that the ability to stimulate NO/cGMP pathway by NtB would result in improving relaxation of penile smooth muscle, mainly when defective NO/cGMP is related to ED, specially in diabetic ED (Angulo et al., Diabetes exacerbates the functional deficiency of NO/cGMP pathway associated with erectile dysfunction in human corpus cavernosum and penile arteries, J Sex Med 2010, 7:758-768).

2) NtB Relaxes Rat Corpus Cavernosum.

FIG. 19 shows the capacity of NtB to concentration-dependently relax corpus cavernosum strips from rat penis. This further suggests a potential capacity of NtB to improve penile smooth muscle relaxation.

Finally, FIG. 20 shows a schematic vision of the rationale and evidences stated above.

### EXAMPLE 8

### Methods Used in the Examples

Cell Proliferation Assays (FIGS. 7, 8, 16, and 17):

Human androgen-sensitive prostate cancer cell line, LNCaP, and androgen-insensitive prostate cancer cell line, PC-3 were cultured in RPMI-1640 medium supplemented with 2 mM L-glutamine and 10% fetal bovine serum (FBS). For proliferation assays LNCaP and PC-3 cells were seeded at 15,000 cells/well density in 24-well plates with complete medium (with 10% FBS). Cells were let to attach for 20 h and then, the culture medium was exchanged for fresh serum-free medium containing the respective treatments (androgens and test compounds). Viable cells content in each well was determined 72 h afterwards.

Proliferation was determined by the XTT cell viability assay (Cell Proliferation Kit II (XTT), Sigma-Aldrich, St. Louis, Mo., USA). This is a colorimetric assay that analyzes the number of viable cells by the cleavage of tetrazolium salts added to the culture medium. The cleavage product of tetrazolium salt XTT, in contrast to MTT, is soluble in water. The tetrazolium salt XTT is cleaved to formazan by a complex cellular mechanism. This bioreduction occurs in viable cells only. Cells grown in culture plates were incubated with the XTT labeling mixture (XTT plus electron coupling reagent in 50:1 ratio) for 4 hours. After this incubation period, the amount of formazan dye formed was quantitated using a plate reader. The measured absorbance at 490 nm directly correlates to the number of viable cells.

Prostatic-Specific Antigen (PSA) Determinations (FIGS. 9 and 18):

For this purpose, LNCaP cells were seeded at 25,000 cells/well in 24-well plates and let to grow in complete medium (10% FBS) at 80% confluence, approximately, that is typically achieved after 48 h. The culture medium was then exchanged for fresh serum-free medium containing the respective treatments (androgens and/or NtB and/or lycopene). Conditioned media was collected 24 h afterwards and PSA concentrations were determined by using a colorimetric ELISA kit following manufacturer's instructions (Ray Biotech, Norcross, Ga., USA).

Organ Bath Experiments with Human Prostate and Bladder Tissues (FIGS. 10, 12, 19, and 20):

Specimens of human prostate and bladder neck were obtained from patients undergoing suprapubic adenomectomy (Millin's approach) for benign prostatic hyperplasia (BPH). Tissue specimens were placed in ice-cold M-400 solution (pH 7.4; 400 mOsm/kg. Composition in w/v: 4.19% mannitol, 0.2% KH₂PO₄, 0.97% K₂HPO₄·3 H₂O, 0.11% KCl and 0.08% NaHCO₃) and transported to the laboratory for utilization within 24 h (Angulo et al., Tadalafil enhances the inhibitory effects of tamsulosin on neurogenic contractions of human prostate and bladder neck, J Sex Med 2012, 9:2293-2306; La Fuente et al., Stimulation of large-conductance calcium-activated potassium channels inhibits neurogenic contraction of human bladder from patients with urinary symptoms and reverses acetic acid-induced bladder hyperactivity in rats, Eur J Pharmacol. 2014, 735:68-76). The study complied with Spanish regulation regarding human tissue collection, conservation and elimination and the protocols were approved by the Ethics Committees at the Hospitals where the tissues were collected in both Spain and Portugal. Patients provided their informed consent for being included in the study.

Human prostate and bladder neck specimens were cleaned of fat and connective tissue and cut into strips for organ bath assays. Strips of human prostate and bladder neck were mounted on force transducers in 8 ml organ baths containing Krebs-Henseleit solution (KHS) which is comprised of the following composition (mM): NaCl 119, KC1 4.6, CaCl ₂ 1.5, MgC1₂ 1.2, NaHCO₃ 24.9, glucose 11, KH₂PO₄ 1.2, EDTA 0.027 at 37° C. continuously bubbled with 95% O₂/5% CO₂ mixture to maintain a pH of 7.4. Prostate and bladder strips were submitted to a resting tension of 1.5 g and then left for equilibration for 90 min with extensive washouts. Tissues were subsequently exposed to 125 mM K⁺ (equimolar substitution of NaCl for KCl in KHS) and contractile response was measured to check functionality. The relaxant effect induced by increasing doses of the NtB extract or the vehicle (distilled water) was evaluated in human prostate and bladder neck strips precontracted with 3-10 μM norepinephrine (NE) (80% of K⁺-induced contraction, approximately) (FIG. 10). Relaxations of human prostate and bladder neck induced by PDES inhibition were evaluated by adding increasing concentrations of tadalafil (1 nM to 100 μM) on strips precontracted with NE and previously treated with NtB (10 mg/ml) or vehicle (distilled water) (FIG. 12). For evaluation of the effects of lycopene on NtB-induced relaxation, human prostate strips were treated with lycopene (0.25 mg/ml) or vehicle (dimethylsulfoxide). Thirty minutes afterwards, strips were contracted with NE and exposed to increasing concentrations of NtB (0.1 mg/ml to 30 mg/ml) (FIG. 19). Concentration-response (relaxation) curves to lycopene (0.025 to 0.5 mg/ml) were also determined after NE-induced contraction in human bladder neck (FIG. 20A).

Measurement of Cyclic GMP in Human Prostate and Bladder Neck (FIG. 11):

Human prostate and bladder neck strips were immersed in 8 ml organ chambers containing KHS, maintained at 37° C. and aerated with 5% CO₂/95% O₂, pH of 7.4. Tissues were incubated for 15 minutes with NtB (30 mg/ml) or vehicle, then immediately frozen in liquid nitrogen and stored at −80° C. until extraction for cyclic nucleotide assay. Tissues were extracted by homogenization in 6% trichloroacetic acid followed by ether (H₂O-saturated) extraction and lyophilization. Finally, cyclic GMP was determined by ELISA (Cayman Chemical Co, Ann Arbor, Mich., USA) (Angulo et al., Tadalafil enhances the inhibitory effects of tamsulosin on neurogenic contractions of human prostate and bladder neck, J Sex Med 2012, 9:2293-2306).

Evaluation of Acute and Chronic NtB on Urodynamic Parameters in BPH Rat Model (FIGS. 13, 14 and 15)

For the BPH rat model, procedures have been approved by the Ethics Committee for Animal Experimentation of the Hospital Universitario Ramón y Cajal. Six weeks old male Sprague-Dawley rats were anesthetized with ketamine and diazepam and, through a transversal incision on scrotum, testicles were surgically removed for castration. After ligature of vessels and closure of the incision with sutures, rats received an intramuscular injection of analgesic (metamizol; 200 mg/kg) and antibiotic (gentamicin; 10 mg/kg) and were let to recover for one week. Castrated rats were daily injected with testosterone (3 mg/kg, s.c.) for 5 weeks. For evaluation of chronic effects, after three weeks of testosterone treatment, NtB (30 mg/kg) or the vehicle (tap water) was daily administered by oral gavage for two additional weeks together with testosterone daily injections (see scheme in FIG. 24). This rat model of BPH displays alteration of urodynamic parameters after 21 days of daily testosterone injections (Liu et al., Amlodipine alone or combined with terazosin improves lower urinary tract disorder in rat models of benign prostatic hyperplasia, BJU Int 2009, 104:1752-1757).

Cystometries were performed as previously described (La Fuente et al., Stimulation of large-conductance calcium-activated potassium channels inhibits neurogenic contraction of human bladder from patients with urinary symptoms and reverses acetic acid-induced bladder hyperactivity in rats, Eur J Pharmacol. 2014, 735:68-76). Rats were anesthetized with urethane (1.2 g/kg; i.p.) and left carotid artery was cannulated to continuously register systemic blood pressure by means of a pressure transducer connected to a MacLab data acquisition system (ADlnstruments, Castle Hill, Australia). Heart rate was obtained from blood pressure signal. The urinary bladder was exposed and a polyethylene catheter was placed into the bladder lumen and fixed with a suture. The catheter was connected to a pressure transducer and to the data acquisition system to register intravesical pressure. Intravesical catheter was also connected to an infusion pump (Harvard Apparatus, Harvard, MA, USA). After an equilibrium period, a 20 min continuous infusion of bladder with saline (0.9% NaCl; 5 ml/h) was performed. Micturition frequency, micturition volume, time of infusion to first micturition, intravesical pressure (IVP, bladder pressure), and threshold pressure (IVP reached just before micturition reflex) were determined. For evaluation of acute effects of NtB, after an equilibration period, NtB (100 mg/kg) was intraduodenally injected to anesthetized vehicle-treated BPH rats and, 20 min later cystometries were again performed and urodynamic parameters determined.

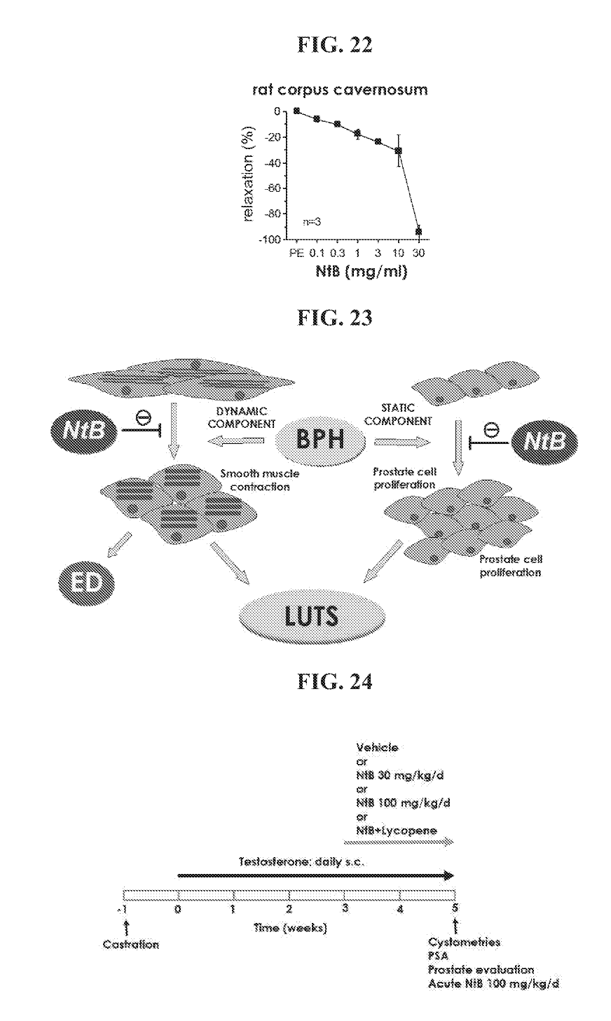

Evaluation of NtB on Relaxation of Rat Corpus Cavernosum (FIG. 22):

Experiments were conducted as previously described (Martinez-Salamanca et al., α1A-Adrenergic Receptor Antagonism Improves Erectile and Cavernosal Responses in Rats With Cavernous Nerve Injury and Enhances Neurogenic Responses in Human Corpus Cavernosum From Patients With Erectile Dysfunction Secondary to Radical Prostatectomy, J Sex Med 2016, 13(12):1844-1857). Rats were killed by anesthetic overdose and exsanguinated by carotid arteries section. The penises were immediately excised. Two strips of corpus cavernosum (RCC) from each penis were carefully dissected through respective longitudinal incisions along the tunica albuginea. Strips of CC were mounted on force transducers in 8 ml organ baths (37° C.) containing KHS continuously bubbled with 95% O₂/5% CO₂ (pH of 7.4). CC strips were submitted to 0.3 g of resting tension. After 60 min equilibration period, tissues were exposed to 75 mM K⁺ and contraction was measured. Relaxation responses were evaluated by cumulative additions of NtB (0.1 to 30 mg/ml) on PE-contracted RCC strips.

### EXAMPLE 9

### Effects of the Combination of Lycopene and N. tangutorum Bobr Extract on Urodynamics and Prostate Growth in Rats with Testosterone-Induced BPH

The present disclosure demonstrates that N. tangutorumj Bobr extract causes effective concentration-dependent relaxation of human prostate and bladder neck and cGMP accumulation in these tissues. In addition, this fruit extract inhibits proliferation of androgen-sensitive (LNCaP) and androgen-insensitive (PC-3) prostate cancer cells while not influencing androgen-stimulated prostatic specific antigen production by human prostate cancer cells. Thus, potential activity of N. tangutorum Bobr extract to relieve benign prostatic hyperplasia (BPH)-induced symptoms would be based on two effects: i) relaxation of prostate and bladder neck smooth muscle and ii) reducing prostate size. However, addition of an additional compound with anti-androgen activity as lycopene would increase the potential activity of N. tangutorum Bobr extract to relieve benign prostatic hyperplasia (BPH)-induced symptoms. In this sense, lycopene has been shown to inhibit prostate cell proliferation and PSA production by reducing androgen-stimulated signaling (Herzog et al., 2005; Liu et al., 2008; Zhang et al., 2010). Combination of N. tangutorum Bobr extract and lycopene could represent a strategy for treating both dynamic and structural alterations responsible for BPH-induced symptoms.

One aim of this example is to evaluate the in vivo effects of the combination of lycopene and N. tangutorum Bobr extract (NtB) on urinary symptoms and prostate growth in rats with testosterone-induced BPH.

For the BPH rat model, six weeks old male Sprague-Dawley rats are anesthetized with ketamine and diazepam and, through a transversal incision on scrotum, testicles are surgically removed for castration. After ligature of vessels and closure of the incision with sutures, rats receive an intramuscular injection of analgesic (metamizol; 200 mg/kg) and antibiotic (gentamicin; 10 mg/kg) and are let to recover for one week. Sham operated rats (without testicle removal) are used as control group. Castrated rats receive daily injections of testosterone (3 mg/kg, s.c.) for three weeks. At this time, NtB (30 mg/kg), NtB plus lycopene (3 mg/kg), or the vehicle is daily administered by oral gavage for two additional weeks together with testosterone daily injections (see scheme in FIG. 24). This rat model of BPH displays alteration of urodynamic parameters after 21 days of daily testosterone injections (Liu et al., 2009).

Cystometries were performed as described in Example 8 ([00380]).

Prostatic-Specific Antigen (PSA) Determinations. Once cystometric evaluations are completed, blood is collected by cardiac puncture. Blood are immediately centrifuged and serum are obtained, frozen and stored at −80° C. until determinations. PSA concentrations are determined by using a colorimetric ELISA kit following manufacturer's instructions.

Prostate Evaluation. After blood collection, rats are sacrificed by anesthetic overdose and prostates are immediately removed, cleaned of fat or surrounding tissue and weighed. As a measure of prostate hypertrophy, the ratio between prostate weight and body weight was calculated (mg of prostate weight/100 g of body weight).

Statistical Analyses. Complete concentration-response curves are compared by a two-factor analysis of variance (ANOVA). Other data are compared by t-test or one-factor ANOVA followed by Student-Newmann-Keuls test (multiple comparisons). StatView and GraphPad InStat software for Apple computers are used for statistical analyses. A p<0.05 are considered significant.

Protocols:

The N. tangutorum Bobr. extract is herein denominated as NtB.

1. Evaluation of oral administration of NtB and NtB plus lycopene on urodynamic parameters in BPH rats. Cystometries are performed in sham operated rats (control group) and testosterone-injected castrated rats orally treated with vehicle, NtB 30 mg/kg, NtB 100 mg/kg or NtB 30 mg/kg plus lycopene 3 mg/kg. Six to 8 animals with valid cystometric determinations for each of 5 groups are required.

2. Evaluation of acute administration of NtB on urodynamic parameters in BPH rats. Cystometries are performed in testosterone-injected castrated rats and, after establishing urodynamic parameters in control conditions, NtB 100 mg/kg or vehicle are intraduodenally administered. Sixty min after administration, urodynamic parameters are again determined. Five to 6 animals with valid cystometric determinations for each of 2 groups are required.

3. Evaluation of oral administration of NtB and NtB plus lycopene on serum levels of PSA in BPH rats. PSA are determined in serum obtained from sham operated rats (control group) and testosterone-injected castrated rats orally treated with vehicle, NtB 30 mg/kg, NtB 100 mg/kg or NtB 30 mg/kg plus lycopene 3 mg/kg. Sera are obtained from rats of first protocol.

4. Evaluation of oral administration of NtB and NtB plus lycopene on prostate size and histology in BPH rats. Prostate volume and weight, histological structure, and expression of PCNA and α-SMA are determined in sham operated rats (control group) and testosterone-injected castrated rats orally treated with vehicle, NtB 30 mg/kg, NtB 100 mg/kg or NtB 30 mg/kg plus lycopene 3 mg/kg. Prostate is obtained from rats of first protocol.

A total amount of 42-56 animals are used. It is estimated that 6 months are required for completion of the study.

The foregoing examples are embodiments of the present disclosure, which is not limited thereto. Any change, modification, replacement, combination and simplification made within the spirit essence and principle of the present disclosure shall be regarded as falling into the protection scope of the present disclosure as equivalents.

1. A method for obtaining an extract from Nitraria tangutorum Bobr., comprising:
(1) mixing a sample of Nitraria tangutorum Bobr. fruit with an alcohol (e.g., ethanol) solution, e.g., from about 30% (v/v) to about 95% (v/v), such as about 65% (v/v);
(2) incubating the mixture under a temperature between about 10° C. and about 60° C., such as between about room temperature (e.g., about 18° C.) and about 55° C.;
(3) obtaining a liquid phase sample from the mixture, and optionally filtering the liquid phase sample, removing the alcohol from the liquid phase sample, and/or concentrating the liquid phase sample, wherein optionally the remaining sample comprises solid, semi-solid, and/or liquid matter; and
(4) allowing evaporation of the alcohol and/or water from the liquid phase sample, wherein the resulting sample after evaporation comprises an extract comprising one or more ingredients from Nitraria tangutorum Bobr.

2. The method of claim 1, wherein in the mixing step, the ratio between the sample weight and the alcohol solution volume is between about (1 g):(3 mL) and about (1 g):(10 mL), optionally between about (1 g):(3 mL) and about (1 g):(5 mL).

3. The method of claim 1 or 2, wherein the incubating step is carried out for between about 1 hour and about 2 hours, or more than about 2 hours.

4. The method of any one of claims 1-3, wherein the incubating step is carried out while stirring the mixture, e.g., for extraction of the ingredient into the liquid phase sample.

5. The method of any one of claims 1-4, wherein in the obtaining step, the liquid phase sample is extracted from the mixture, and the remaining sample comprises mostly solid matter.

6. The method of any of claims 1-5, wherein the sample of Nitraria tangutorum Bobr. fruit comprises an intact fruit, flesh of the fruit, fruit pulp, a seed, a fresh fruit, a dried fruit, a chilled fruit, a frozen fruit, a preserved fruit, a milled fruit, a minced fruit, a crushed fruit, a granulated fruit, a powered fruit, or any combination thereof.

7. The method of any of claims 1-6, wherein the sample of Nitraria tangutorum Bobr. fruit is a dried sample, a semi-wet sample, or a wet sample.

8. The method of any of claims 1-7, further comprising obtaining the sample of Nitraria tangutorum Bobr. fruit before the mixing step.

9. The method of any of claims 1-8, further comprising cutting, shredding, mincing, or milling the sample of Nitraria tangutorum Bobr. fruit before the mixing step.

10. The method of any of claims 1-9, further comprising purifying or isolating the extract and/or the ingredient from the resulting sample after evaporation.

11. The method of any of claims 1-10, further comprising drying the resulting sample after evaporation, wherein optionally a powder extract comprising the ingredient is obtained.

12. The method of any of claims 1-11, wherein steps (1)-(3) are repeated one, two, or more times before step (4).

13. The method of any one of claims 1-11, wherein after obtaining the liquid phase sample, the method further comprises:
(a) obtaining from the mixture a remaining sample which comprises mostly solid matter, wherein the mixture is a first mixture;
(b) mixing the remaining sample with an alcohol (e.g., ethanol) solution, e.g., from about 30% (v/v) to about 95% (v/v), such as about 65% (v/v), wherein optionally the ratio between the weight of the remaining sample and the alcohol solution volume is between about (1 g):(3 mL) and about (1 g):(10 mL) and optionally between about (1 g):(3 mL) and about (1 g):(5 mL), to obtain a second mixture;
(c) incubating the second mixture under a temperature between about room temperature (e.g., about 18° C.) and about 55° C.;
(d) obtaining a second liquid phase sample from the second mixture, wherein the liquid phase sample from the first mixture is a first liquid phase sample;
(e) combining the first and second liquid phase samples, and optionally filtering the combined liquid phase sample; and
(f) allowing evaporation from the combined liquid phase sample, wherein the resulting sample after evaporation comprises the extract comprising one or more ingredients from Nitraria tangutorum Bobr.

14. The method of claim 13, wherein steps (a)-(d) are repeated one, two, or more times before step (e), or wherein steps (a)-(e) are repeated one, two, or more times before step (f).

15. The method of any one of claims 1-14, further comprising pressure treatment and/or ultra-sonication during the mixing, incubating, and/or obtaining step.

16. The method of claim 15, wherein the pressure is between about 30 MPa and about 150 MPa, e.g., 50 MPa.

17. The method of claim 15 or 16, wherein the ultra-sonication has a power between about 100 W and about 10,000 W, e.g., about 600 W.

18. The method of any of claims 1-17, further comprising rotatable evaporating the liquid phase sample at a temperature of between about 20° C. and about 70° C. (such as about 55° C.) and/or a reduced pressure (such as between about −5 MPa and about 5 MPa), e.g., in order to remove a solvent in the liquid phase sample.

19. The method of any of claims 1-18, further comprising passing the liquid phase sample through a first chromatography column, which optionally comprises a macroporous resin adsorption column, e.g., in order to remove a sugar from the liquid phase sample.

20. The method of any of claims 1-19, further comprising passing the liquid phase sample through a second chromatography column, which optionally comprises a macroporous resin adsorption column, e.g., in order to increase the anthocyanin and/or polyphenol concentration in the liquid phase sample.

21. The method of claim 20, wherein the first and second chromatography columns are the same or different.

22. The method of any of claims 19-21, wherein before the chromatography, a part or substantially all of the alcohol is removed (e.g., by drying or rotatable evaporating the alcohol) from the liquid phase sample, which is then re-dissolved in water.

23. The method of any of claims 19-22, wherein the chromatography comprises eluting the column, e.g., with an alcohol solution of about 50% (v/v) to about 100% (v/v), such as about 95% (v/v).

24. The method of any of claims 1-23, wherein the allowing step comprises freeze-drying (lyophilization) and/or spray-drying the resulting sample to obtain the extract.

25. A method for obtaining an extract from Nitraria tangutorum Bobr., comprising:
(1) extracting a sample of Nitraria tangutorum Bobr. fruit in a first supercritical fluid extraction device, to obtain an oil-like extract and a remaining sample;
(2) mixing the remaining sample with an entrainer and extracting the mixture in a second supercritical fluid extraction device, to obtain a liquid phase sample; and
(3) allowing evaporation from the liquid phase sample, wherein the resulting sample after evaporation comprises an extract comprising one or more ingredients from Nitraria tangutorum Bobr.

26. The method of claim 25, wherein the first supercritical extraction is carried out under a pressure between about 20 MPa and about 35 MPa, a temperature between about 35° C. and about 55° C., and/or a supercritical fluid flow rate between 0.5 L/min and about 3 L/min (such as 2 L/min) and optionally between about 1 L/min and about 3 L/min.

27. The method of claim 25 or 26, wherein the first supercritical extraction is carried out for between about 1 hour and about 3 hours, optionally between about 2 hours and about 3 hours.

28. The method of any of claims 25-27, wherein the second supercritical extraction is carried out under a pressure between about 20 MPa and about 35 MPa, a temperature between about 35° C. and about 55° C., a supercritical fluid flow rate of about 1 L/min, and/or an entrainer flow rate between about 0.2 mL/min and about 1.0 mL/min.

29. The method of any of claims 25-28, wherein the second supercritical extraction is carried out for between about 1 hour and about 3 hours.

30. The method of any of claims 25-29, wherein the remaining sample in the mixing step is a defatted remaining sample.

31. The method of any of claims 25-30, wherein between about 5 g and about 500 kg (such as about 10 g, 200 kg, or 500 kg) of the sample of Nitraria tangutorum Bobr. fruit is used for each extraction.

32. The method of any of claims 25-31, wherein the supercritical fluid comprises CO₂.

33. The method of any of claims 25-32, wherein the first and second supercritical fluid extraction devices are the same or different.

34. The method of any of claims 25-33, wherein the entrainer comprises an alcohol (e.g., ethanol) and/or water.

35. The method of any of claims 25-34, wherein the entrainer comprises between about 35% (v/v) and about 95% (v/v) of an alcohol such as ethanol.

36. The method of any of claims 25-35, wherein the ratio between the volume of the entrainer and the weight of the remaining sample in the mixing step is about (1 mL):(1 g) or less than about (1 mL):(1 g), such as less than about (0.1 mL):(1 g), between about (0.1 mL):(1 g) and about (0.5 mL):(1 g), or between about (0.5 mL):(1 g) and about (1 mL):(1 g).

37. The method of any of claims 25-36, wherein the remaining sample is partially or fully infiltrated with the entrainer in the mixing step.

38. The method of any of claims 25-37, wherein in the mixing step, the remaining sample, after being infiltrated with the entrainer and prior to the second supercritical fluid extraction, is statically soaked in the supercritical fluid (e.g., CO₂), e.g., for about 30 minutes.

39. The method of any of claims 25-38, wherein between about 5 g and about 250 kg (such as about 5 g, 100 kg, or 250 kg) of the remaining sample in the mixing step is used for the second supercritical fluid extraction.

40. The method of any of claims 25-39, wherein the sample of Nitraria tangutorum Bobr. fruit comprises an intact fruit, flesh of the fruit, fruit pulp, a seed, a fresh fruit, a dried fruit, a chilled fruit, a frozen fruit, a preserved fruit, a milled fruit, a minced fruit, a crushed fruit, a granulated fruit, a powered fruit, or any combination thereof.

41. The method of any of claims 25-40, wherein the sample of Nitraria tangutorum Bobr. fruit is a dried sample, a semi-wet sample, or a wet sample.

42. The method of any of claims 25-41, further comprising obtaining the sample of Nitraria tangutorum Bobr. fruit before the extracting step.

43. The method of any of claims 25-42, further comprising cutting, shredding, mincing, or milling the sample of Nitraria tangutorum Bobr. fruit before the extracting step.

44. The method of any of claims 25-43, further comprising purifying or isolating the extract and/or the ingredient from the resulting sample after evaporation.

45. The method of any of claims 25-44, further comprising drying the resulting sample after evaporation, wherein optionally a powder extract or a semi-solid extract comprising the ingredient is obtained.

46. The method of any of claims 25-45, wherein the drying comprises freeze-drying (lyophilization) and/or spray-drying.

47. The method of any of claims 25-46, further comprising rotatable evaporating the liquid phase sample before drying, at a temperature between about 35° C. and about 55° C. (e.g., at about 50° C.) and/or under a pressure of about −0.09 MPa, e.g., in order to remove a solvent in the liquid phase sample.

48. The method of any of claims 45, wherein the drying is carried out at a temperature of about −50° C. and/or under a pressure of about 5 Pa.

49. The method of any of claims 1-45, wherein the one or more ingredients comprise one or more anthocyanins and/or one or more polyphenols.

50. The liquid phase sample, the remaining sample, the resulting sample, the extract, and/or the one or more ingredients from Nitraria tangutorum Bobr. produced by the method of any of claims 1-24 and 49.

51. The liquid phase sample, the remaining sample, the resulting sample, the extract, and/or the one or more ingredients from Nitraria tangutorum Bobr. of claim 50 for use in the treatment and/or prevention of a condition or disease in a subject in need thereof, optionally without adversely affecting an arterial pressure such as the mean arterial pressure and/or heart rate of the subject.

52. Use of the liquid phase sample, the remaining sample, the resulting sample, the extract, and/or the one or more ingredients from Nitraria tangutorum Bobr. of claim 50 in the manufacture of a medicament for treating and/or preventing a condition or disease in a subject in need thereof.

53. The oil-like extract, the remaining sample, the liquid phase sample, the resulting sample, the extract, and/or the one or more ingredients from Nitraria tangutorum Bobr. produced by the method of any of claims 25-49.

54. The oil-like extract, the remaining sample, the liquid phase sample, the resulting sample, the extract, and/or the one or more ingredients from Nitraria tangutorum Bobr. of claim 53 for use in the treatment and/or prevention of a condition or disease in a subject in need thereof.

55. Use of the oil-like extract, the remaining sample, the liquid phase sample, the resulting sample, the extract, and/or the one or more ingredients from Nitraria tangutorum Bobr. of claim 53 in the manufacture of a medicament for treating and/or preventing a condition or disease in a subject in need thereof.

56. A pharmaceutical composition comprising the liquid phase sample, the remaining sample, the resulting sample, the extract, and/or the one or more ingredients from Nitraria tangutorum Bobr. of claim 50, and optionally a pharmaceutically acceptable excipient and/or diluent.

57. A pharmaceutical composition comprising the oil-like extract, the remaining sample, the liquid phase sample, the resulting sample, the extract, and/or the one or more ingredients from Nitraria tangutorum Bobr. of claim 53, and optionally a pharmaceutically acceptable excipient and/or diluent.

58. The pharmaceutical composition of claim 56 or 57, further comprising lycopene or a composition or extract comprising lycopene, saw palmetto or an extract thereof, pumpkin seed or an extract thereof (such as a protein extract), Selenium (Se), Lyceum ruthenicum or an extract thereof, black tomato or an extract thereof, lentinan, Pleurotus ostreatus polysaccharide, agaric polysaccharide, Flammulina velutipes polysaccharide, spirulina or an extract thereof, lutein, zeaxanthin, and/or astaxanthin.

59. The pharmaceutical composition of any of claims 56-58, which is in a human dosage form.

60. The pharmaceutical composition of any of claims 56-59, for use in the treatment and/or prevention of a condition and/or disease in a subject in need thereof, optionally without adversely affecting an arterial pressure such as the mean arterial pressure and/or heart rate of the subject.

61. The pharmaceutical composition of claim 60, wherein the condition and/or disease comprises a lower urinary tract symptom due to benign prostatic hypertrophy (e.g., BPH/LUTS), macular degeneration, and a cancer.

62. The pharmaceutical composition of claim 61, wherein the macular degeneration is associated with radiation (e.g., from a radioactive material or from an ultraviolet light), or wherein the macular degeneration is age-related.

63. The pharmaceutical composition of claim 61, wherein the cancer is prostate cancer.

64. The pharmaceutical composition of claim 61 or 63, wherein the cancer is sensitive to an androgen, such as testosterone or dihydrotestosterone.

65. The pharmaceutical composition of claim 61 or 63, wherein the cancer is insensitive to an androgen, such as testosterone or dihydrotestosterone.

66. The pharmaceutical composition of any one of claims 63-65, wherein the liquid phase sample, the remaining sample, the resulting sample, the extract, and/or the one or more ingredients from Nitraria tangutorum Bobr. in the pharmaceutical composition exert an anti-cancer effect, such as an anti-proliferative effect, optionally without adversely affecting an arterial pressure such as the mean arterial pressure and/or heart rate of the subject.

67. The pharmaceutical composition of claim 66, wherein the anti-proliferative effect is independent of androgen signaling.

68. The pharmaceutical composition of claim 66 or 67, wherein the liquid phase sample, the remaining sample, the resulting sample, the extract, and/or the one or more ingredients from Nitraria tangutorum Bobr. in the pharmaceutical composition do not interfere with androgen-stimulated prostate specific antigen (PSA) production in the cancer.

69. The pharmaceutical composition of any one of claims 56-68, wherein the liquid phase sample, the remaining sample, the resulting sample, the extract, and/or the one or more ingredients from Nitraria tangutorum Bobr. in the pharmaceutical composition relaxes a tissue or an organ.

70. The pharmaceutical composition of claim 69, wherein the organ is a prostate or bladder and the tissue is a human prostate tissue or a human bladder neck.

71. The pharmaceutical composition of claim 69 or 70, wherein the tissue or organ is from a subject suffering from a lower urinary tract symptom due to benign prostatic hypertrophy, or a subject suspected of suffering from the lower urinary tract symptom due to benign prostatic hypertrophy, wherein the benign prostatic hypertrophy is optionally induced by an androgen.

72. The pharmaceutical composition of any one of claims 69-71, wherein the tissue or organ is relaxed due to stimulation of NO and/or cGMP (nitric oxide and/or cyclic GMP) production.

73. The pharmaceutical composition of any one of claims 56-72, further comprising a PDES inhibitor such as tadalafil.

74. The pharmaceutical composition of claim 58, wherein the lycopene or a composition or extract comprising lycopene has an anti-cancer effect, such as an anti-proliferative effect, optionally wherein the lycopene or composition or extract comprising lycopene inhibits or reduces prostate specific antigen (PSA) production in a cancer such as prostate cancer.

75. The pharmaceutical composition of claim 74, wherein the anti-proliferative effect is dependent on androgen signaling, and lycopene inhibits androgen-induced proliferation of an androgen-sensitive cancer cell but not proliferation of an androgen-insensitive cancer cell.

76. The pharmaceutical composition of any one of claims 58, 74, and 75, wherein the lycopene or a composition or extract comprising lycopene enhances an anti-cancer effect of the liquid phase sample, the remaining sample, the resulting sample, the extract, and/or the one or more ingredients from Nitraria tangutorum Bobr.

77. The pharmaceutical composition of claim 76, wherein the anti-cancer effect is an anti-proliferative effect on an androgen-sensitive cancer cell, such as a human prostate cancer cell.

78. The pharmaceutical composition of claim 58, wherein the lycopene or a composition or extract comprising lycopene relaxes a tissue or an organ.

79. The pharmaceutical composition of claim 78, wherein the lycopene or a composition or extract comprising lycopene does not interfere with relaxation of the organ or tissue caused by the liquid phase sample, the remaining sample, the resulting sample, the extract, and/or the one or more ingredients from Nitraria tangutorum Bobr. in the pharmaceutical composition.

80. The pharmaceutical composition of any of claims 56-79, for improving a vascular function, blood circulation, a cerebral function, and/or an immune function, and/or for preventing and/or alleviating a symptom and/or consequence of erectile dysfunction, hypertension, arteriosclerosis, thrombosis, fatigue, cerebral apoplexy, and/or stroke.

81. The pharmaceutical composition of claim 80, wherein the pharmaceutical composition stimulates NO and/or cGMP production and optionally comprises a PDES inhibitor such as tadalafil.

82. The pharmaceutical composition of claim 80 or 81, wherein the pharmaceutical composition relaxes a tissue or an organ.

83. The pharmaceutical composition of claim 82, wherein the organ is a penis and the tissue is a smooth muscle, such as a corpus cavernosum.

84. The pharmaceutical composition of any of claims 56-83, for alleviating a side effect of a therapy, such as treatment with an anticancer agent.

85. The pharmaceutical composition according to any of claims 56-84, wherein said pharmaceutical composition is prepared in a form selected from the group consisting of a liquid, a powder a tablet, a granule, a pill, a capsule (e.g., a hard capsule or a soft capsule), an oral cream, a paste, a decoction, a syrup, a wine, a distillate, and any combination thereof.

86. The pharmaceutical composition according to any of claims 56-85, in a dosage form for oral, gastrointestinal, topical, mucosal, intravenous, intradermal, subcutaneous, or intramuscular administration.

87. A method of treating and/or preventing a condition and/or disease in a subject in need thereof, comprising administering to the subject a pharmaceutically effective dose of:
(i) the liquid phase sample, the remaining sample, the resulting sample, the extract, and/or the one or more ingredients from Nitraria tangutorum Bobr. of claim 50;
(ii) the oil-like extract, the remaining sample, the liquid phase sample, the resulting sample, the extract, and/or the one or more ingredients from Nitraria tangutorum Bobr. of claim 53; and/or
(iii) the pharmaceutical composition according to any of claims 56-86.

88. The method of claim 87, used in combination with another therapy or regimen for treating and/or preventing the condition and/or disease.

89. The method of claim 88, which is used before, during, and/or after the other therapy or regimen, or in an alternating fashion with the other therapy or regimen.

90. The method of any one of claims 87-89, further comprising administering to the subject a pharmaceutically effective dose of lycopene or a composition or extract comprising lycopene.

91. The method of any one of claims 87-90, further comprising administering to the subject a pharmaceutically effective dose of a PDES inhibitor such as tadalafil.

92. The method of any one of claims 87-91, wherein the condition and/or disease is selected from the group consisting of a lower urinary tract symptom due to benign prostatic hypertrophy (e.g., BPH/LUTS), macular degeneration, a cancer such as prostate cancer (including androgen-sensitive or androgen-insensitive prostate cancer) or bladder cancer, erectile dysfunction, hypertension, arteriosclerosis, thrombosis, fatigue, cerebral apoplexy, and stroke, or any combination thereof, optionally wherein the administration does not adversely affect an arterial pressure such as the mean arterial pressure and/or heart rate of the subject.

93. A food additive, comprising:
(i) the liquid phase sample, the remaining sample, the resulting sample, the extract, and/or the one or more ingredients from Nitraria tangutorum Bobr. of claim 50;
(ii) the oil-like extract, the remaining sample, the liquid phase sample, the resulting sample, the extract, and/or the one or more ingredients from Nitraria tangutorum Bobr. of claim 53; and/or
(iii) the pharmaceutical composition according to any of claims 56-86.

94. A health supplement, comprising:
(i) the liquid phase sample, the remaining sample, the resulting sample, the extract, and/or the one or more ingredients from Nitraria tangutorum Bobr. of claim 50;
(ii) the oil-like extract, the remaining sample, the liquid phase sample, the resulting sample, the extract, and/or the one or more ingredients from Nitraria tangutorum Bobr. of claim 53; and/or
(iii) the pharmaceutical composition according to any of claims 56-86.

95. The food additive of claim 93 or health supplement of claim 94, in a form selected from the group consisting of a liquid, a powder a tablet, a granule, a pill, a capsule (e.g., a hard capsule or a soft capsule), an oral cream, a paste, a decoction, a syrup, a wine, a distillate, and any combination thereof.
